(19) United States
(12) Patent Application Publication
(10) Pub. No.: US 2025/0276945 A1
(43) Pub. Date: Sep. 4, 2025

(54) MICROBIAL FERTILIZER

(71) Applicant: ELM DIRT, LLC, Grandview, KS (US)

(72) Inventor: Lauren Cain, Grandview, MO (US)

(21) Appl. No.: 18/592,821

(22) Filed: Mar. 1, 2024

(51) Int. Cl. C05F 17/20; C05B 17/00; C12N 1/14; C12N 1/20

(52) U.S. Cl. CPC C05F 17/20; C05B 17/00; C12N 1/14; C12N 1/20

(57) ABSTRACT

The present disclosure pertains to microbial fertilizer compositions, and methods for their production and utilization. Specifically, the disclosure encompasses fertilizer compositions incorporating a stable culture of live microorganisms. The methods described herein involve the manufacture of these fertilizer compositions, as well as their application for various purposes, including but not limited to, plant nutrition, promotion of plant growth, resistance against pests, and soil conditioning.

### TECHNICAL FIELD

The present disclosure relates generally to microbial fertilizer compositions, along with methods of making and using the same. More particularly, but not exclusively, the present disclosure relates to fertilizer compositions comprising a stable culture of live microorganisms, methods of making the fertilizer compositions, and methods of using the same for purposes such as plant nutrition, plant growth promotion, pest resistance, and soil conditioning.

### BACKGROUND

Fertilizers are frequently used in agriculture to provide crops with key nutrients such as nitrogen, phosphorous, and potassium. Use of fertilizers increases plant growth rate, increases yield, conditions the soil, and can assist in pest deterrence or resistance. Increasingly, there is an interest in using select microorganisms in combination with or simultaneously with conventional fertilizers. It has been found that the supply of certain types of microorganisms to the soil can increase fertilizer uptake by plants roots, increase organic matter catabolism, and further assist in mitigating the soil depletion which often occurs due to the use of conventional chemical fertilizers.

However, incorporating microorganisms into a stable fertilizer poses several challenges. Many liquid fertilizers, particularly those containing nitrogen, are toxic to bacteria. Some existing products solve this issue by storing and applying the fertilizer and microorganisms to the soil separately. This solution is undesirable because it requires the use of multiple formulations and results in increased labor due to multiple applications of products.

Furthermore, cultures of microorganisms generally demonstrate poor stability and a limited shelf life. The microorganisms are extremely sensitive to environmental changes, including changes in temperature, UV light exposure, pH, and oxygen availability. This sensitivity leads to a significant decline in populations and an increase in materials waste. Current microbial products are typically fermented in large manufacturing facilities, then concentrated such that population numbers are increased above the label values, thereby allowing the manufacturer to account for the death of viable organisms during shipping and storage. Other attempted solutions to reduce loss of microbial viability include the use of various transport and storage media to reduce the activity of the microorganisms. However, these solutions still result in significant loss of microbe population and do not solve the problem of incorporating a stable microbial culture into a nutrient-rich fertilizer.

Thus, there is still a need to address the low levels of initial activity and/or low percent cell viability of such microorganisms. There is correspondingly a need to stabilize cultures of microorganisms useful for soil fertilization.

There is also a need for compositions and methods of incorporating active microorganisms into fertilizer compositions that do not result in microorganism inactivation or the undesirable generation of gas byproducts.

Overall, there is a need for systems and methods for providing user friendly, reliable, and viable microbial fertilizer compositions.

These and other objects, advantages, and features of the present disclosure will become apparent from the following specification taken in conjunction with the claims set forth herein.

### BRIEF SUMMARY

It is an advantage of the compositions and methods described herein that the microbial consortia in the compositions are stabilized and further that the fertilizer compositions and methods of use provide a variety of benefits to treated plants, plant tissues, plant parts, and soils, for example, improved nutrient uptake, improved growth rate, increased pest resistance, increased heartiness (e.g., drought resistance, lodging resistance), better yield, and overall better health.

Disclosed herein are microbial fertilizer compositions comprising one or more digestates; a seaweed; one or more cellulose sources; one or more nutrient supplements; one or more humic substances; a probiotic; and a microbial consortium.

In an embodiment, the one or more digestates comprise vermicompost, seabird guano, or a combination thereof.

In an embodiment, the seaweed comprises green algae, brown algae, red algae, or a combination thereof. In a further embodiment, the brown algae comprises kelp.

In an embodiment, the one or more cellulose sources comprise alfalfa, comfrey, nettles, yarrow, yucca, agave, or a combination thereof.

According to an embodiment, the one or more nutrient supplements comprise bone meal, fish meal, magnesium sulfate, calcium sulfate, silicon dioxide, potassium silicate, calcium silicate silicic acid, or a combination thereof.

In an embodiment, the one or more humic substances comprise humins, humic acids, fulvic acids, or a combination thereof.

In an embodiment, the probiotic comprises lactic acid bacteria, yeast, phototrophic bacteria, or a combination thereof.

In some embodiments, the microbial fertilizer composition comprises from about 5 wt. % to about 75 wt. % of the one or more digestates, from about 5 wt. % to about 45 wt. % of the seaweed, from about 0.001 wt. % to about 30 wt. % of the one or more cellulose sources, from about 0.01 wt. % to about 90 wt. % of the nutrient supplements, from about 0.01 wt. % to about 0 wt. % of the of the humic substances, from about 0.01 wt. % to about 5 wt. % of the probiotic, and from about 0.01 wt. % to about 5 wt. % of the microbial consortium.

In further embodiments, the composition further comprises an additive comprising an anti-corrosion agent, anti-caking agent, stabilizer, anti-freeze, anti-foam agent, sticking agent, spreading agent, wetting agent, drift control agent, complexing agent, softening agent, an immune system enhancer, an additional source of primary nutrients or secondary nutrients or micronutrients, or a combination thereof.

Also disclosed herein are methods of generating a microbial consortium comprising culturing one or more microorganisms comprising proteobacteria, firmicutes, bacteroidota, actinobacteria, campylobacterota, verrucomicrobiota, desulfobacterota, ascomycota, mortierellomycota, basidiomycota, mucoromycota, olpidiomycota, or a combination thereof to form a microbial culture; adding a probiotic to the microbial culture; and fermenting the one or more microorganisms for between about 24 hours and about 48 hours to form a microbial consortium; and optionally separating the microbial consortium into solid and liquid fractions.

Additionally disclosed are methods of making a microbial fertilizer composition comprising combining one or more digestates with a seaweed, a cellulose source, one or more nutrient supplements, one or more humic substances, and a probiotic to form a base fertilizer composition; combining the base fertilizer composition with the microbial consortium described herein to form a microbial fertilizer; and fermenting the microbial fertilizer.

In an embodiment, the methods further comprise a step of aerating the microbial fertilizer during the fermenting step.

In one embodiment, the fermenting step lasts for between about 24 hours and about 48 hours.

Also disclosed are methods of treating a soil, plant, plant tissue, or plant part comprising contacting a soil, plant, plant tissue, plant part, or a combination thereof with a microbial fertilizer composition comprising one or more digestates, a seaweed, one or more cellulose sources, one or more nutrient supplements, one or more humic substances, a probiotic, and a microbial consortium to form a treated product; and optionally, repeating the contacting.

In an embodiment, the plant part comprises foliage, a stem, root, seedling, seed, or a combination thereof, and wherein the treated product comprises a treated soil, a treated plant, a treated plant tissue, or a treated plant part.

In an embodiment, the contacting lasts for between about 1 minute to about 24 hours.

In an embodiment, the period of time in between the repeating of the contacting step is between 24 hours and 12 weeks.

In an embodiment, the microbial consortium described herein, including each embodiment described in the brief summary, comprises NRRL No. XXXXX, NRRL No.

YYYYY, NRRL No. ZZZZZ, Oryzomicrobium terrae, Cloacibacterium sp., Acinetobacter brisouii, Dysgonomonas mossii, Acinetobacter calcoaceticus, Acetobacteroides hydrogenigenes, Prevotella sp., Sedimentibacter sp., Azotobacter chroococcum, Prevotella paludivivens, Comamonas testosteroni, Lentilactobacillus sp., Acetobacter syzygii, Leuconostoc mesenteroides, Bacteroides sp., Arcobacter butzleri, Xanthomonas massiliensis, Microvirgula aerodenitrificans, Lactococcus lactis, Bifidobacterium mongoliense, Dechlorosoma suillum, Clostridium sp., Citrobacter freundii, Bacteroides luti, Bifidobacterium psychraerophilum, Pseudomonas sp., Lactobacillus paracasei, Salmonella enterica, Dysgonomonas sp., Desulfosporosinus sp., Acinetobacter junii, Comamonas sp., Azoarcus indigens, Chthoniobacter sp., Clostridium magnum, Lactobacillus harbinensis, Neobacillus drentensis, Clostridium methoxybenzovorans, Desulfovibrio sp., Colidextribacter sp., Comamonas aquatica, Lachnoclostridium sp., Clostridium saccharolyticum, Desulfosporosinus hippei, Sporomusa sp., Fonticella sp., Anaerovorax sp., Acinetobacter haemolyticus, Lactobacillus perolens, Desulfitobacterium sp., Mobilitalea sp., Desulfitobacterium dichloroeliminans, Achromobacter xylosoxidans, Clostridium sporosphaeroides, Acinetobacter sp., Rhodobacter sp., Lactobacillus bifermentans, Paenirhodobacter enshiensis, Hydrogenoanaerobacterium sp., Clostridium beijerinckii, Stenotrophomonas maltophilia, Elizabethkingia meningoseptica, Acinetobacter kookii, Stenotrophomonas acidaminiphila, Clostridium viride, Enterococcus casseliflavus, Sporomusa ovata, Desulfotomaculum defluvii, Pedobacter tournemirensis, Nitrosotenuis sp., Oscillibacter sp., Chryseobacterium taihuense, Ruminiclostridium sp., Ruminococcus sp., Anaerocolumna sp., Escherichia coli, Bacillus sp., Amnipila sp., Reyranella sp., Leuconostoc pseudomesenteroides, Comamonas terrigena, Citrobacter amalonaticus, Enterococcus italicus, Lactobacillus vaccinostercus, Butyricicoccus sp., Monoglobus sp., Kosakonia oryzae, Micropepsis sp., Clostridium tunisiense, Dechlorosoma sp., Ilyobacter delafieldii, Lacticaseibacillus sp., Udaeobacter sp., Delftia sp., Ruminiclostridium hungatei, Sporomusa silvacetica, Nitrosocosmicus oleophilus, Desulfotomaculum sp., Desulfosporomusa polytropa, Acidipropionibacterium acidipropionici, Caproiciproducens sp., Pandoraea pnomenusa, Actinomyces sp., Herbaspirillum huttiense, Phenylobacterium sp., Papillibacter sp., Saccharibacillus sp., Pseudomonas otitidis, Schaalia turicensis, Desulfosporosinus fructosivorans, Novosphingobium resinovorum, Lactobacillus delbrueckii, Pseudomonas putida, Anaerocolumna xylanovorans, Pseudomonas aeruginosa, Acinetobacter variabilis, Caulobacter sp., Clostridium subterminale, Acinetobacter ursingii, Oxobacter sp., Acetanaerobacterium sp., Clostridium intestinale, Lactobacillus pentosus, Sporomusa malonica, Rhodovastum sp., Clostridium homopropionicum, Paracoccus sp., Clostridium malenominatum, Anaerostignum sp., Aeromonas hydrophila, Desulfosporosinus meridiei, Acetobacter sp., Pseudarthrobacter oxydans, Azospirillum sp., Citrobacter sp., Pseudomonas stutzeri, Herbinix sp., Stenotrophomonas sp., Serratia marcescens, Acinetobacter radioresistens, Desulfosporosinus youngiae, Desulfofarcimen sp., Trabulsiella sp., Oxalophagus oxalicus, Clostridium tyrobutyricum, Enterococcus sp., Tabrizicola sp., Hydrogenophaga sp., Novosphingobium sp., Neorhizobium alkalisoli, Methylophilus sp., Desulfobulbus sp., Geminisphaera sp., Paludibacter sp., Solimonas flava, Flavobacterium sp., Pleomorphomonas sp., Sphingobium sp., Shewanella dokdonensis, Klebsiella pneumoniae, Rhizobium sp., Pseudacidovorax intermedius, Magnetospirillum sp., Siphonobacter aquaeclarae, Oleomonas sagaranensis, Riegeria sp., Quatrionicoccus sp., Paenirhodobacter sp., Ideonella dechloratans, Roseomonas sp., Xanthobacter polyaromaticivorans, Thiobacillus thioparus, Ancalomicrobium sp., Uliginosibacterium sp., Anaerosporobacter sp., Pseudomonas oleovorans, Devosia insulae, Agrobacterium tumefaciens, Erythrobacter mathurensis, Propionivibrio sp., Lacunisphaera sp., Chitinophaga pinensis, Xanthobacter autotrophicus, Mucilaginibacter litoreus, Dyadobacter fermentans, Brevundimonas aurantiaca, Bosea sp., Chitinophaga sancti, Xanthobacter flavus, Sphingobium yanoikuyae, Desulfovibrio vulgaris, Azoarcus olearius, Catellibacterium terrae, Rhodovarius lipocyclicus, Aquaspirillum polymorphum, Roseomonas rubra, Starkeya novella, Paracoccus yeei, Gracilibacter sp., Dechlorosoma suillum, Aeromonas sp., Acinetobacter johnsonii, Anaerotaenia torta, Parabacteroides chartae, Pedobacterrhizosphaerae, Anaerocolumna cellulosilytica, Rhizobium undicola, Shinella zoogloeoides, Bdellovibrio sp., Propionicimonas sp., Lachnotalea sp., Bosea thiooxidans, Variovorax ginsengisoli, Kaistia hirudinis, Zoogloea sp., Kaistia dalseonensis, Opitutus sp., Kaistia sp., Terrimicrobium sacchariphilum, Mycolicibacterium mucogenicum, Asticcacaulis sp., Rhizobium gallicum, Cellulosimicrobium sp., Prosthecobacter debontii, Mycobacterium gilvum, Sphingomonas wittichii, Cereibacter changlensis, Pseudomonas mendocina, Variovorax sp., Pseudaeromonas pectinilytica, Cupriavidus campinensis, Bacillus circulans, Fibrisoma sp., Moraxella osloensis, Ensifer adhaerens, Rhizobium rhizoryzae, Novosphingobium aromaticivorans, Shinella fusca, Roseomonas eburnea, Roseomonas cervicalis, Acinetobacter soli, Rhodoplanes roseus, Paenibacillus ihuae, Caulobacter mirabilis, Enterobacter sp., Flavobacterium akiainvivens, Pseudomonas alcaligenes, Paenibacillus graminis, Marinomonas sp., Xinfangfangia soli, Imtechium assamiensis, Raoultella planticola, Roseomonas aestuarii, Legionella lytica, Novosphingobium sediminicola, Yersinia pestis, Raoultella ornithinolytica, Enterobacter hormaechei, Mycoplana sp., Pseudomonas nitroreducens, Rhizobium petrolearium, Shinella kummerowiae, Propionispora vibrioides, Enterobacter cancerogenus, Ochrobactrum intermedium, Klebsiella sp., Acinetobacter tandoii, Cloacibacterium haliotis, Lactiplantibacillus sp., Yersinia bercovieri, Pelosinus sp., Acinetobacter lwoffii, Anaerospora sp., Lactobacillus coryniformis, Pseudomonas psychrotolerans, Agrobacterium albertimagni, Acinetobacter venetianus, Cyberlindnera jadinii, Penicillium herquei, Williopsis sp., Mortierella hyalina, Pichia kudriavzevii, Mucor circinelloides, Candida tropicalis, Preussia flanaganii, Mortierella sp., Penicillium sp., Penicillium melinii, Mortierella gamsii, Starmera stellimalicola, Purpureocillium lavendulum, Mucor irregularis, Monocillium indicum, Penicillium griseofulvum, Solicoccozyma aeria, Mortierella alpina, Monocillium mucidum, Mortierella ambigua, Tetracladium sp., Tausonia pullulans, Trichoderma piluliferum, Stropharia coronilla, Gymnostellatospora japonica, Aspergillus caninus, Meyerozyma caribbica, Penicillium bilaiae, Trichoderma harzianum, Pseudogymnoascus roseus, Penicillium oxalicum, Talaromyces marne ei, Phaeosphaeriopsis sp., Penicillium brevicompactum, Alternaria longissima, Candida parapsilosis, Pseudostrickeria sp., Allophoma tropica, Leptosphaeria maculans, Trichoderma pubescens, Cortinarius helvolus, Mortierella sarnyensis, Pyrenochaetopsis leptospora, Exophiala sp., Pseudothielavia terricola, Cladosporium herbarum, Trichoderma asperellum, Aspergillus fumigatus, Alternaria eichhorniae, Talaromyces piceae, Penicillium levitum, Talaromyces sp., Chrysosporium lobatum, Didymella exigua, Aspergillus chlamydosporus, Penicillium roseopurpureum, Subramaniula asteroides, Aspergillus pseudodeflectus, Penicillium citrinum, Chrysosporium pseudomerdarium, Mortierella rishikesha, Neobulgaria sp., Curvularia verruculosa, Clavaria sp., Plectosphaerella cucumerina, Mortierella minutissima, Penicillium parviverrucosum, Panaeolus fimicola, Psathyrella ichnusae, Arachnomyces gracilis, Solicoccozyma terrea, Mortierella antarctica, Coniothyrium cerealis, Neodactylaria sp., Penicillium pimiteouiense, Penicillium simplicissimum, Humicola olivacea, Arachnomyces kanei, Mortierella lignicola, Thelebolus globosus, Bipolaris microlaenae, Penicillium erubescens, Polyschema sclerotigenum, Phaeosphaeria sp., Sarocladium strictum, Penicillium restrictum, Fusarium proliferatum, Penicillium argentinense, Alternaria metachromatica, Coniothyrium sp., Talaromyces purpureogenus, Aspergillus niger, Lachnum sp., Gymnopus sp., Penicillium rubens, Alternaria photistica, Sporormiella megalospora, Didymella pinodes, Phallus rugulosus, Coniochaeta canina, Cutaneotrichosporon jirovecii, Aspergillus ochraceus, Penicillium catenatum, Microscypha sp., Sebacina sp., Talaromyces stollii, Oidiodendron cereale, Hannaella oryzae, Furcasterigmium furcatum, Fusarium solani, Clonostachys rosea, Aspergillus wentii, Kernia pachypleura, Olpidium brassicae, Penicillium steckii, Byssochlamys lagunculariae, Acrocalymma vagum, Cephalotrichum microsporum, Stemphylium vesicarium, Leohumicola verrucosa, Aspergillus subversicolor, Wallemia sebi, Trichosporon asahii, Rhizoctonia solani, Actinomucor elegans, Scedosporium dehoogii, Scedosporium boydii, Gibellulopsis serrae, Vishniacozyma victoriae, Cunninghamella echinulata, Chaetosphaeronema sp., Chrysosporium sp., Filobasidium magnum, Epicoccum thailandicum, Aspergillus sp., Oidiodendron echinulatum, Penicillium polonicum, Oedocephalum nayoroense, Fusarium oxysporum, Hirsutella minnesotensis, Aspergillus flavus, Penicillium atrovenetum, Chrysosporium synchronum, Hymenoscyphus menthae, Talaromyces radicus, Mortierella elongata, Microascus trigonosporus, Humicola fuscoatra, Vishniacozyma tephrensis, Aspergillus terreus, Neosetophoma samarorum, Aureobasidium pullulans, Wallemia canadensis, Circinella muscae, Botrytis caroliniana, Aspergillus penicillioides, Aspergillus rugulosus, Preussia sp., Malbranchea flocciformis, Malbranchea cinnamomea, Microdochium sp., Fusarium pseudensiforme, Chaetomium acropullum, Vishniacozyma globispora, or a combination thereof.

These and/or other objects, features, advantages, aspects, and/or embodiments will become apparent to those skilled in the art after reviewing the following brief and detailed descriptions of the drawings. The present disclosure encompasses both combinations of disclosed aspects and/or embodiments and/or reasonable modifications not shown or described.

### BRIEF DESCRIPTION OF THE FIGURES

FIG. 1A shows a basil plant before treatment with the fertilizer compositions described herein.

FIG. 1B shows a basil plant after treatment with the fertilizer compositions described herein.

FIG. 2A shows a first succulent plant before treatment with the fertilizer compositions described herein.

FIG. 2B shows the first succulent plant after treatment with the fertilizer compositions described herein.

FIG. 3A shows a second succulent plant before treatment with the fertilizer compositions described herein.

FIG. 3B shows the second succulent plant after treatment with the fertilizer compositions described herein.

FIG. 4A shows a Ficus plant before treatment with the fertilizer compositions described herein.

FIG. 4B shows a Ficus plant after treatment with the fertilizer compositions described herein.

### DETAILED DESCRIPTION

The present disclosure relates generally to microbial fertilizer compositions, along with methods of making and using the same. More particularly, but not exclusively, the present disclosure relates to fertilizer compositions comprising a stable culture of live microorganisms, methods of making the fertilizer compositions, and methods of using the same for purposes such as plant nutrition, plant growth promotion, pest resistance, and soil conditioning.

The embodiments of this disclosure are not limited to particular types of compositions or methods, which can vary. It is further to be understood that all terminology used herein is to describe particular embodiments only and is not intended to be limiting in any manner or scope. For example, as used in this specification and the appended claims, the singular forms “a,” “an” and “the” can include plural referents unless the context indicates otherwise. Unless indicated otherwise, “or” can mean any one alone or any combination thereof, e.g., “A, B, or C” means the same as any of A alone, B alone, C alone, “A and B,” “A and C,” “B and C” or “A, B, and C.” Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, a description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 1½, and 4¾ This applies regardless of the breadth of the range.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The terms “a,” “an,” and “the” include both singular and plural referents.

The term “or” is synonymous with “and/or” and means any one member or combination of members of a particular list.

The term “about,” as used herein, refers to variation in the numerical quantity that can occur, for example, through typical measuring techniques and equipment, with respect to any quantifiable variable, including, but not limited to, mass, volume, time, temperature, pH, reflectance, whiteness, etc. Further, given solid and liquid handling procedures used in the real world, there is certain inadvertent error and variation that is likely through differences in the manufacture, source, or purity of the ingredients used to make the compositions or carry out the methods and the like. The term “about” also encompasses amounts that differ due to different equilibrium conditions for a composition resulting from a particular initial mixture. The term “about” also encompasses these variations. Whether or not modified by the term “about,” the claims include equivalents to the quantities.

The term “actives” or “percent actives” or “percent by weight actives” or “actives concentration” are used interchangeably herein and refer to the concentration of those ingredients involved in cleaning expressed as a percentage minus inert ingredients such as water or salts.

The term “mass percent,” “percent by mass,” “% by mass,” “w/w %” and variations thereof, as used herein, refer to the concentration of a substance as the weight of that substance divided by the total mass of the composition and multiplied by 100. It is understood that, unless specified otherwise, “percent,” “%,” and the like are intended to be synonymous with “mass percent,” etc. and further that “mass percent” and all variations thereof may be used interchangeably with “wt. %,” “percent by weight,” “% by weight,” and “weight percent.”

As used herein, the term “substantially free” refers to compositions completely lacking the component or having such a small amount of the component that the component does not affect the performance of the composition. The component may be present as an impurity or as a contaminant and shall be less than 0.5 wt. %. In another embodiment, the amount of the component is less than 0.1 wt. % and in yet another embodiment, the amount of component is less than 0.01 wt. %.

As used herein, the term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae. As used herein, the term “microbe” is synonymous with microorganism. The term “bacteria” as used herein encompasses bacteria, bacteria-like organisms, and their equivalents, including actinomycetes.

The term “vitamin” refers to organic compounds that are essential or useful in varying quantities for the nutrition of plants. Vitamins are generally classified as either water-soluble or non-water-soluble (e.g., fat soluble). The term “mineral” refers to an inorganic substance essential or useful in varying quantities for the nutrition of plants.

The methods, systems, apparatuses, and compositions disclosed herein may comprise, consist essentially of, or consist of the components and ingredients described herein as well as other ingredients not described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

The “scope” of the present disclosure is defined by the claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

### Fertilizer Compositions

Example fertilizer compositions according to the present disclosure are provided in the following tables. The fertilizer compositions may be provided in any suitable form, for example as a liquid (e.g., an aqueous solution or suspension) including liquid concentrates and diluted “ready to use” use solutions or a solid, including a powder, pellet, or block. When the fertilizer compositions are incorporate into a use solution, the compositions are diluted with water at a ratio of between about 1:1 to 1:128. For example, for houseplants, the compositions may be diluted at a ratio of between about 1 ounce to about 2 ounces of composition per gallon of water. For garden plants, shrubs, bushes, potted trees, and lawns the compositions may be diluted at a ratio of between about 2 ounces to about 4 ounces per gallon of water. For in-ground trees, the compositions may be diluted at a ratio of between about 6 ounces to about 10 ounces of composition per each inch in the tree diameter with one gallon of water. The fertilizer compositions disclosed herein may also be incorporated into another substance, such as soil, to generate a pre-fertilized soil mixture. Tables 1A to 2B provide example formulations of the fertilizer compositions. Specifically, these tables detail the raw mass of components utilized in each Example formulation, as well as analogous mass % ranges that correspond to the raw mass of the compositions. Thus, the fertilizer compositions (e.g., Example 1) may be described in terms of its raw mass, or various suitable mass % ranges of each of the components.

Additionally, each of the fertilizer compositions described herein, such as those in Tables 1A-1B may be provided individually or in combination. That is, the fertilizer compositions described herein may comprise or consist of the formulation Example 1 in Table 1 alone, and/or the compositions may comprise or consist of a combination of formulations, such as a combination of Example 2 and Example 4. When provided in combination, the formulations may be prepared in any suitable ratio, such as 10:90, 20:80, 25:75, 50:50, and the like.

TABLE 1A
Example 1

| | Amount | Example 1.1 | Example 1.2 |
|---|---|---|---|
| Component | (mass) | (mass %) | (mass %) |
| Digestate | 25-45 kg | 35-75% | 50-60% |
| Seaweed | 15-20 kg | 15-45% | 20-35% |
| Cellulose Source | 5-15 kg | 1-30% | 10-20% |
| Nutrient Supplement(s) | 350-1450 g | 0.01-5% | 0.1-3% |
| Humic Substance(s) | 340-530 g | 0.01-5% | 0.1-2% |
| Probiotic | 0.1-0.3 kg | 0.01-3% | 0.01-2% |
| Microbial Consortia | 20-80 g | 0.01-3% | 0.01-1% |

TABLE 1B
Example 2

| | | Example | Example |
|---|---|---|---|
| | Amount | 2.1 | 2.2 |
| Component | (mass) | (mass %) | (mass %) |
| Digestate | 25-45 kg | 35-75% | 50-60% |
| Seaweed | 15-20 kg | 15-45% | 20-35% |
| Cellulose Source | 5-15 kg | 1-30% | 10-20% |
| First Nutrient Supplement | 0.1-1 kg | 0.01-5% | 0.1-2% |
| Second Nutrient Supplement | 250-450 g | 0.01-5% | 0.1-2% |
| First Humic Substance | 300-450 g | 0.01-5% | 0.1-2% |
| Second Humic Substance | 40-80 g | 0.001-3% | 0.01-1% |
| Probiotic | 0.1-0.3 kg | 0.01-3% | 0.01-2% |
| Microbial Consortia | 20-80 g | 0.01-3% | 0.01-1% |

TABLE 2A
Example 3

| | | Example | Example |
|---|---|---|---|
| | Amount | 3.1 | 3.2 |
| Component | (mass) | (mass %) | (mass %) |
| Digestate(s) | 5-70 kg | 5-50% | 10-45% |
| Seaweed | 5-25 kg | 5-25% | 10-20% |
| Cellulose Source | 1-500 g | 0.001-5% | 0.001-2% |
| Nutrient Supplement(s) | 10-90 kg | 20-60% | 25-55% |
| Humic Substance(s) | 1-8 kg | 1-10% | 1-8% |
| Probiotic | 0.1-0.3 kg | 0.01-3% | 0.01-2% |
| Microbial Consortia | 20-80 g | 0.01-3% | 0.01-1% |

TABLE 2B
Example 4

| | | | Example | Example |
|---|---|---|---|---|
| | Amount | | 4.1 | 4.2 |
| Component | (mass) | | (mass %) | (mass %) |
| First Digestate | 10-45 | kg | 15-40% | 20-35% |
| Second Digestate | 1-15 | kg | 0.1-20% | 1-10% |
| Seaweed | 5-25 | kg | 5-25% | 10-20% |
| Cellulose Source | 1-500 | g | 0.001-5% | 0.001-2% |
| First Nutrient Supplement | 15-50 | kg | 20-55% | 0.1-2% |
| Second Nutrient Supplement | 1-20 | kg | 1-25% | 1-15% |
| Third Nutrient Supplement | 0.5-10 | kg | 0.5-10% | 1-8% |
| Fourth Nutrient Supplement | 0.1-1 | kg | 0.01-5% | 0.01-3% |
| Fifth Nutrient Supplement | 0.1-1 | kg | 0.01-5% | 0.1-3% |
| Humic Substance | 1-8 | kg | 1-10% | 1-8% |
| Probiotic | 0.1-0.3 | kg | 0.01-3% | 0.01-2% |
| Microbial Consortia | 20-80 | g | 0.01-3% | 0.01-1% |

### Digestate

The fertilizer compositions described herein preferably comprise one or more sources of digestate, also referred to as digested organic matter. Digested organic matter is the product of the breakdown of a source of organic matter by living organisms, whether unicellular or multicellular. Digestates are rich in plant nutrients as they retain nutrients from the input raw material and break said nutrients into a readily available form. Examples of suitable types of digestate include, without limitation, livestock digestate (e.g., cow manure, pig manure), animal digestate (e.g., seabird digestate/guano) and vermicompost.

Worm castings, also referred to as vermicast or vermicompost, are the end product of the breakdown of organic matter by worms, usually white worms, red wigglers, or earthworms. The worms grind and evenly mix minerals, nutrients, and other organic matter such that they are readily available for plants. Vermicompost also functions as a soil conditioner by improving aeration and enriching soil with microorganisms and further improving water retention capabilities of the soil.

Livestock and animal digestate similarly function as a growth aid and a source of readily available and slow-release nutrients and minerals. Key nutrients and minerals include, without limitation, phosphorus, potassium, nitrogen, and carbon.

In an embodiment, the fertilizer compositions include at least one digestate, preferably vermicompost. In another embodiment, the fertilizer compositions include at least two digestates, preferably vermicompost and seabird guano. The one or more digestates may be present, individually or in sum, in an amount of between about 0.1% w/w to about 80% w/w, including between about 35% w/w and about 75% w/w, and between about 50% w/w and about 60% w/w of the fertilizer composition, inclusive of all integers within these ranges. By raw mass, the fertilizer compositions may include one or more digestates, either individually or in sum, in an amount of between about 5 kg to about 100 kg, inclusive of all integers within this range.

### Seaweed and Algae

The fertilizer compositions described herein may comprise one or more seaweed and/or algae. Algae can break down chemical fertilizers in the soil and water and deliver those nutrients in immediately bioavailable form to plants. Algae in the soil surface layers utilize their photosynthetic capabilities to convert carbon dioxide, nutrients, and inorganic nitrogen into cells by means of energy derived from sunlight. Such minerals and nutrients are more readily available for plants. Suitable algae include, but are not limited to Chlorophyta or green algae, Cyanophyta or blue-green algae, Bacillariophyta or diatoms, and Xanthophyta or yellow-green algae.

Seaweed, such as kelp, are multicellular marine algae containing between 60-70 essential minerals and trace elements, as well as vitamins, natural chelating agents, and amino acids that are beneficial for agriculture. Kelp in particular contains magnesium, calcium, iron, copper, potassium, zinc and natural sea salt, along with vitamins A, B, C and E, and beta-carotene. Kelp is also an excellent source of cytokinins and auxins, both of which stimulate plant growth. Suitable types of seaweed include macroalgae such as green algae (Chlorophyta), brown algae (Phaeophyta), and red algae (Rhodophyta). In a preferred embodiment, the fertilizer compositions include a brown algae. In a further preferred embodiment, the brown algae comprises kelp. Any species or combination of species may be utilized, for example bull kelp, giant kelp, southern kelp, or sugarwack. The kelp may be provided in any suitable form, for example as a liquid solution, liquid concentrate, or a solid (e.g., kelp meal).

The one or more seaweed and/or algae may be present in an amount of between about 5% w/w to about 55% w/w, including between about 15% w/w and about 45% w/w, and between about 20% w/w, and between about 20% w/w and about 35% w/w of the fertilizer composition, inclusive of all integers within these ranges. By raw mass, the fertilizer compositions may include seaweed and/or algae in an amount of between about 5 kg to 30 kg, inclusive of all integers within this range.

### Cellulose Source

The fertilizer compositions described herein may also comprise one or more sources of cellulose. Preferably, the cellulose source also functions as a source of nutrients and minerals, such as nitrogen and trace materials. Suitable source of cellulose include, but are not limited to, a plant material or tissue such as rice hulls, rice straw, wheat straw, sorghum sudan straw, barley straw, rye straw, oat straw, rye straw, corn straw, alfalfa, bentgrass hay, softwood sawdust, hardwood sawdust, sunflower seed shells, almond hulls, vetch hay, foxtail grass hay, beardgrass hay, whiskey grass hay, bluestem hay, signal grass, running grass, buffelgrass, lovegrass, bowgrass, hindigrass, bluegrass, crabgrass, couchgrass, barnyard grass, antelopegrass, cupgrass, whipgrass, cogongrass, centipedegrass, sesagrass, armgrass, panicgrass, witchgrass, sweetgrass, millet, torpedograss, ticklegrass, switchgrass, buffalograss, dallisgrass, paspalum, knotgrass, vaseygrass, pennisetum, itchgrass, pigeongrass, bristlegrass, Saint Augustine grass, tasselgrass, goatgrass, quackgrass, slender foxtail, windgrass, Downey Brome, fingergrass, rhodesgrass, bermudagrass, crowfoot grass, goosegrass, stinkgrass, velvetgrass, Hares Tall grass, canarygrass, smutgrass, sisal, sansevieria, pineapple, agave, yucca, wild garlic, nutsedge, clove, comfrey, nettles, yarrow, or a combination thereof. The cellulose source may be provided in any suitable form, for example as a hay, a powder, a meal, or other solid, or as a liquid (e.g., a concentrate or a solution).

In a preferred embodiment, the cellulose source comprises alfalfa, comfrey, nettles, yarrow, yucca, agave, or a combination thereof. Particularly preferred are cellulose sources that contain one or more of the following traits: have and make available key nutrients and mineral (e.g., nitrogen and trace minerals), have the fatty acid growth stimulant tricontanol, have bioactive protein amino acids and peptides, improve soil permeability, improve water and nutrient uptake, improve heartiness/pest resistance, and support microbial populations (e.g., by functioning as a source of complex sugars). In an embodiment, the fertilizer compositions comprise at least one source of cellulose and in a preferred embodiment, the cellulose source comprises alfalfa. In another embodiment, the fertilizer compositions comprise at least two sources of cellulose, and in a preferred embodiment, the cellulose sources comprise alfalfa and yucca.

The one or more cellulose sources may be present in an amount of between about 0.0001% w/w to about 45% w/w, including between about 1% w/w to about 30% w/w, between about 20% w/w to about 35% w/w, and between about 0.001% w/w to about 5% w/w, inclusive of all integers within these ranges. By raw mass, the fertilizer compositions may include a cellulose source in an amount of between about 0.0001 kg to about 25 kg, inclusive of all integers within this range.

### Humic Substances

The fertilizer compositions of the present disclosure preferably include one or more humic substances. Humic substances are heterogenous organic compounds resulting from the decomposition of plant and animal residues. They are found in abundance in soil, sediment, and water. Humic substances comprise approximately 75% of the organic matter in most soils. Humic substances can be classified into humic acids (which are insoluble below pH 2), fulvic acids (which are soluble at any pH), and humin (which is insoluble in water). Because of the complexity and irregularity of humic substances, they are typically not defined by their molecular structure(s) but are rather characterized by average properties. Among the most important of these properties are the prevalence of aromatic structures, which absorb light, induce a variety of photochemical reactions, and are involved in adsorption and aggregation; and the presence of ionic structures, including carboxylic and phenolic groups, which affect solubility of humic matter, and cause complexation of metals and other substances.

Humins, when present within soil, are the most resistant to decomposition of all the humic substances. Some of the main functions of humins are to improve the soil's water holding capacity, to improve soil structure, to maintain soil stability, to function as a cation exchange system, and to generally improve soil fertility.

Humic acids (including humic powder) comprise a mixture of weak aliphatic and aromatic organic acids, which are not soluble in water under acid conditions (low pH) but are soluble in water under alkaline conditions (high pH). Humic acids consist of the fraction of humic substances that are precipitated from aqueous solution when the pH is decreased below 2. Humic acids readily form salts with inorganic trace mineral elements. Humic acids contain a wide variety of minerals and nutrients in a form that can be readily utilized by plants. They are an excellent source of nitrogen, potassium, and phosphorus, they increase the content of alkali nitrogen, available phosphorous, and available potassium. As a result, humic acids function as an important ion exchange system and aid in improving water and nutrient uptake in plants. Beneficially, humic acids can also improve and support microbial growth.

Fulvic acids are a mixture of weak aliphatic and aromatic organic acids, which are soluble in water at all pH conditions (acidic, neutral and alkaline). Fulvic acids have a relatively small molecular weight and accordingly, they can readily enter plant roots, stems, and leaves. As fulvic acids enter these plant parts, they carry trace minerals from plant surfaces into the plant tissues. Once applied to plant foliage, fulvic acids transport trace minerals directly to metabolic sites in plant cells. Thus, fulvic acids stimulate root growth and improve nutrient uptake in plants.

In an embodiment, the fertilizer compositions include at least one humic substance. In a preferred embodiment, the at least one humic substance comprises humic acid. In another embodiment, the fertilizer compositions include at least two or at least three humic substances. In a preferred embodiment, the at least two humic substances comprise humic acid and fulvic acid. The humic substances may be provided in any suitable form, for example as a powder, as a liquid, in granular form, in a solution, or in a liquid concentrate.

The one or more humic substances may be present in an amount of between about 0.001% w/w to about 20% w/w, including between about 0.01% w/w to about 5% w/w, and between about 1% w/w to about 10% w/w of the fertilizer composition, inclusive of all integers within these ranges. By raw mass, the fertilizer compositions may include one or more humic substances, either individually or in sum, in an amount of between about 0.001 kg to about 10 kg, inclusive of all integers within this range.

### Other Mineral & Nutrient Supplements

In addition to the other components described herein, the fertilizer compositions may optionally further comprise one or more additional mineral or nutrient supplements. The nutrient or mineral supplement may consist essentially of the nutrient or mineral itself (e.g., silicon dioxide) or may be a source of one or more key minerals or nutrients (e.g., bone meal) useful in promoting plant growth, soil quality, drought resistance, pest resistance, or any other useful trait.

Useful mineral and/or nutrient supplements include, but are not limited to activated sewage sludge, aluminum sulfate, ammonium metaphosphate, ammonium nitrate, ammonium nitrate solution, ammonium nitrate-limestone mixtures, ammonium nitrate-sulfate, ammonium phosphate, ammonium phosphate nitrate, ammonium phosphate sulfate, ammonium polysulfide, ammonium sulfate, ammonium sulfate solution, ammonium sulfate-nitrate, ammonium sulfate-urea, ammonium thiosulfate, basic lime phosphate, basic slag, bone black spent, bone meal, raw, bone meal, steamed bone, borax, brucite (magnesium hydroxide), calcium ammonium nitrate, calcium chelate, calcium chloride, calcium metaphosphate, calcium nitrate, calcium nitrate-urea, calcium oxide, calcium sulfate, calcium sulfate, lime sulfur solution, castor pomace, cocoa shell meal, cocoa tankage, colloidal phosphate, compost, copper, copper oxide, copper sulfate, cottonseed meal, diammonium phosphate, dolomite, dolomitic lime, dolomitic & calcitic blends, lime suspensions, Epsom salt (magnesium sulfate as a source of magnesium and sulfur), ferrous ammonium sulfate, ferric oxide, ferric sulfate, fish scrap, fish meal, gypsum (calcium sulfate dihydrate, calcium sulfate), guano, iron, lime, linseed meal, liquid ammonium polyphosphate, limestone, magnesium oxide, magnesium chelate, magnesium nitrate, magnesium phosphate, manganous oxide, manganese agstone, manganese chelate, manganese oxide, manganese slag, manganese sulfate, manure, manure salts, monoammonium phosphate, muriate of potash, naphthenic acid, nitrate of soda, nitric acid, nitrogen, non-lime filler, phosphate rock, phosphatic, phosphoric acid, phosphate, lime-potash mixtures, phosphate product, potash, potash suspensions, potassium carbonate, potassium chelate, potassium magnesium sulfate, potassium metaphosphate, potassium nitrate, potassium sulfate, precipitated phosphate, precipitated phosphate, raw potash, sand, sewage sludge, sewage sludge, silica (silicon dioxide, potassium silicate, calcium silicate, silicic acid) sulfur, sulfur urea, sulfuric acid, tobacco stems, triple superphosphate, urea solution, urea-formaldehyde, water, zinc ammonium sulfate solution, zinc chelate, zinc oxide, zinc oxysulfate, zinc sulfate, or a combination thereof.

The one or more additional nutrient supplements or mineral supplements may be provided in any suitable form, including derivatives, salts, or ionic form, and as a solid, powder, meal, liquid concentrate, solution, crystal, or a combination thereof.

In an embodiment, the one or more nutrient supplements comprises bone meal, fish meal, Epsom salt (as magnesium sulfate), gypsum (as calcium sulfate dihydrate and/or calcium sulfate), silica (as silicon dioxide, potassium silicate, calcium silicate and/or silicic acid), or a combination thereof.

The one or more nutrient supplements may be present in an amount of between about 0.001% w/w to about 65% w/w, including between about 0.1% w/w to about 55% w/w, and between about 1% w/w to about 25% w/w of the fertilizer composition, inclusive of all integers within these ranges. In a preferred embodiment, the compositions described herein further comprise a plant immune system enhancer, such as chitosan. By raw mass, the fertilizer compositions may include one or more nutrient supplements, either individually or in sum, in an amount of between about 0.1 kg to about 100 kg, inclusive of all integers within this range.

### Probiotic

The fertilizer compositions disclosed herein may comprise one or more probiotics or probiotics compositions (i.e., compositions comprising multiple species of probiotic microorganisms). Probiotics are live microorganisms, particular bacteria, yeast, and fungi, that in the context of agriculture convey a variety of benefits to plants. For example, probiotics contribute to enhance nutrient update by breaking down organic matter in the soil and releasing nutrients that are otherwise not readily available to plants. They are also capable of converting atmospheric nitrogen into a readily available form, increasing plant growth and yield. Probiotics also contribute to improved soil structure, disease resistance, and stress tolerance.

Any suitable probiotic or probiotic composition may be used, including commercially available probiotic compositions. Suitable microorganisms include those commonly known phototrophic, lactic acid, probiotic, and sulfide-utilizing microorganisms. More particularly, suitable probiotics may comprise one or more of bacterial species selected from Bacillus subtilis, a Lactobacillus sp., a Bifidobacterium sp., a Lactococcus sp., Streptococcus thermophilus, or a combination thereof. In some embodiments, the probiotic composition contains a yeast microorganism. Yeast microorganisms include genera and species within the Ascomycota phylum, including true yeasts and fission yeasts. Preferred yeast microorganisms may include Saccharomyces genus and combinations thereof. Examples of useful yeast include for example Saccharomyces cerevisiae. Further discussion of probiotic compositions and examples of suitable microorganisms is found in U.S. Pat. No. 11,406,672, which is herein incorporated by reference in its entirety. A particularly preferred probiotic according to the present disclosure comprises lactic acid bacteria, yeast, and phototrophic bacteria. Such a probiotic is available commercially as EM-i®.

The probiotic (whether an individual species of microorganism or a probiotic composition) may be present in an amount of between about 0.001% w/w to about 15% w/w, including between about 0.01% w/w to about 3% w/w, and between about 0.01% w/w to about 2% w/w of the fertilizer composition, inclusive of all integers within these ranges. By raw mass, the fertilizer compositions may include one or more probiotics (whether individual species of microorganisms and probiotic compositions) in an amount between about 10 grams and about 100 grams, wherein the probiotic comprises between about 1 million colony forming units/cc (units/ml) of Lactobacillus casei, inclusive of all integers within this range.

### Stabilized Microbial Consortium

Disclosed herein are stabilized microbial consortia. A microbial consortium comprises a mixture, association, or assemblage of two or more microbial species. The microorganisms in the consortium may interact or affect one another through direct physical contact, through biochemical interactions, or both. Alternatively, microorganisms in the consortium may be metabolically independent.

Also disclosed herein are consortia or fertilizer compositions including two or more (such as 2 or more, 5 or more, 10 or more, 20 or more, or 50 or more) or all of the microorganisms in the stabilized microbial consortium. In some embodiments and as described herein, the fertilized compositions may comprise a defined microbial consortium, for example a consortium including specified microbial species along with additional non-microbial components (for example, nutrient supplements, humic substances, sources of cellulose digestates, etc.). In some examples, the microbial consortium includes aerobic and anaerobic microorganisms.

The stabilized consortia may include one or more of bacteria in the following phyla: Proteobacteria, Firmicutes, Bacteroidota, Actinobacteria, Campylobacterota, or a combination thereof. The stabilized consortia may additionally or alternatively include fungi from the following phyla: Ascomycota, Mortierellomycota, Basidiomycota, Mucoromycota, or a combination thereof.

In an embodiment, the stabilized consortia include one or more of the following bacterial organisms:

TABLE 3
Example Consortium 1-Bacteria

| # | Genus & Species | Cells/ml | Cells/ml Range |
|---|---|---|---|
| 1 | Oryzomicrobium terrae | 7.35E+08 | 6.62E+08-8.08E+08 |
| 2 | Cloacibacterium sp. | 8.42E+07 | 7.58E+07-9.26E+07 |
| 3 | Acinetobacter brisouii | 6.94E+08 | 6.24E+08-7.63E+08 |
| 4 | Dysgonomonas mossii | 7.91E+07 | 7.12E+07-8.70E+07 |
| 5 | Acinetobacter calcoaceticus | 5.94E+08 | 5.35E+08-6.53E+08 |
| 6 | Acetobacteroides hydrogenigenes | 7.65E+07 | 6.88E+07-8.41E+07 |
| 7 | Prevotella sp. | 5.85E+08 | 5.26E+08-6.44E+08 |
| 8 | Sedimentibacter sp. | 7.41E+07 | 6.67E+07-8.15E+07 |
| 9 | Azotobacter chroococcum | 4.62E+08 | 4.16E+08-5.07E+08 |
| 10 | Prevotella paludivivens | 7.28E+07 | 6.55E+07-7.99E+07 |
| 11 | Comamonas testosteroni | 3.15E+08 | 2.83E+08-3.46E+08 |
| 12 | Lentilactobacillus sp. | 6.41E+07 | 5.77E+07-7.05E+07 |
| 13 | Acetobacter syzygii | 2.79E+08 | 2.51E+08-3.07E+08 |
| 14 | Leuconostoc mesenteroides | 6.39E+07 | 5.75E+07-7.03E+07 |
| 15 | Bacteroides sp. | 2.58E+08 | 2.32E+08-2.84E+08 |
| 16 | Arcobacter butzleri | 6.34E+07 | 5.71E+07-6.97E+07 |
| 17 | Xanthomonas massiliensis | 2.25E+08 | 2.03E+08-2.48E+08 |
| 18 | Microvirgula aerodenitrificans | 5.65E+07 | 5.09E+07-6.22E+07 |
| 19 | Lactococcus lactis | 2.05E+08 | 1.85E+08-2.26E+08 |
| 20 | Bifidobacterium mongoliense | 5.53E+07 | 4.98E+07-6.08E+07 |
| 21 | Dechlorosoma suillum | 1.64E+08 | 1.47E+08-1.81E+08 |
| 22 | Clostridium sp. | 4.90E+07 | 4.41E+07-5.39E+07 |
| 23 | Citrobacter freundii | 1.33E+08 | 1.20E+08-1.46E+08 |
| 24 | Bacteroides luti | 4.87E+07 | 4.38E+07-5.36E+07 |
| 25 | Bifidobacterium psychraerophilum | 1.24E+08 | 1.12E+08-1.36E+08 |
| 26 | Pseudomonas sp. | 4.85E+07 | 4.37E+07-5.33E+07 |
| 27 | Lactobacillus paracasei | 9.81E+07 | 8.83E+07-1.08E+08 |
| 28 | Salmonella enterica | 3.89E+07 | 3.50E+07-4.28E+07 |
| 29 | Dysgonomonas sp. | 9.40E+07 | 8.46E+07-1.03E+08 |
| 30 | Desulfosporosinus sp. | 3.87E+07 | 3.48E+07-4.26E+07 |
| 31 | Acinetobacter junii | 3.13E+07 | 2.82E+07-3.44E+07 |
| 32 | Comamonas sp. | 1.04E+07 | 9.36E+06-1.14E+07 |
| 33 | Azoarcus indigens | 3.08E+07 | 2.77E+07-3.39E+07 |
| 34 | Chthoniobacter sp. | 1.03E+07 | 9.27E+06-1.13E+07 |
| 35 | Clostridium magnum | 2.48E+07 | 2.23E+07-2.73E+07 |
| 36 | Lactobacillus harbinensis | 1.03E+07 | 9.27E+06-1.13E+07 |
| 37 | Neobacillus drentensis | 1.76E+07 | 1.58E+07-1.93E+07 |
| 38 | Clostridium methoxybenzovorans | 9.62E+06 | 8.66E+06-1.06E+07 |
| 39 | Desulfovibrio sp. | 1.71E+07 | 1.54E+07-1.88E+07 |
| 40 | Colidextribacter sp. | 8.55E+06 | 7.70E+06-9.40E+06 |
| 41 | Comamonas aquatica | 1.66E+07 | 1.49E+07-1.82E+07 |
| 42 | Lachnoclostridium sp. | 8.55E+06 | 7.70E+06-9.40E+06 |
| 43 | Clostridium saccharolyticum | 1.65E+07 | 1.48E+07-1.81E+07 |
| 44 | Desulfosporosinus hippei | 8.17E+06 | 7.35E+06-8.99E+06 |
| 45 | Sporomusa sp. | 1.58E+07 | 1.42E+07-1.74E+07 |
| 46 | Fonticella sp. | 7.69E+06 | 6.92E+06-8.46E+06 |
| 47 | Anaerovorax sp. | 1.58E+07 | 1.42E+07-1.74E+07 |
| 48 | Acinetobacter haemolyticus | 7.69E+06 | 6.92E+06-8.46E+06 |
| 49 | Lactobacillus perolens | 1.57E+07 | 1.41E+07-1.73E+07 |
| 50 | Desulfitobacterium sp. | 7.26E+06 | 6.53E+06-7.99E+06 |
| 51 | Mobilitalea sp. | 1.56E+07 | 1.40E+07-1.71E+07 |
| 52 | Desulfitobacterium | 6.70E+06 | 6.03E+06-7.37E+06 |
| | dichloroeliminans | | |
| 53 | Achromobacter xylosoxidans | 1.51E+07 | 1.36E+07-1.66E+07 |
| 54 | Clostridium sporosphaeroides | 6.62E+06 | 5.96E+06-7.28E+06 |
| 55 | Acinetobacter sp. | 1.50E+07 | 1.35E+07-1.65E+07 |
| 56 | Rhodobacter sp. | 5.98E+06 | 5.38E+06-6.58E+06 |
| 57 | Lactobacillus bifermentans | 1.45E+07 | 1.31E+07-1.59E+07 |
| 58 | Paenirhodobacter enshiensis | 5.98E+06 | 5.38E+06-6.58E+06 |
| 59 | Hydrogenoanaerobacterium sp. | 1.45E+07 | 1.31E+07-1.59E+07 |
| 60 | Clostridium beijerinckii | 5.86E+06 | 5.27E+06-6.45E+06 |
| 61 | Stenotrophomonas maltophilia | 1.41E+07 | 1.27E+07-1.55E+07 |
| 62 | Elizabethkingia meningoseptica | 5.77E+06 | 5.19E+06-6.35E+06 |
| 63 | Acinetobacter kookii | 1.34E+07 | 1.21E+07-1.47E+07 |
| 64 | Stenotrophomonas acidaminiphila | 5.70E+06 | 5.13E+06-6.27E+06 |
| 65 | Clostridium viride | 1.32E+07 | 1.19E+07-1.45E+07 |
| 66 | Enterococcus casseliflavus | 5.47E+06 | 4.92E+06-6.02E+06 |
| 67 | Sporomusa ovata | 1.30E+07 | 1.17E+07-1.43E+07 |
| 68 | Desulfotomaculum defluvii | 5.34E+06 | 4.81E+06-5.87E+06 |
| 69 | Pedobacter tournemirensis | 1.24E+07 | 1.12E+07-1.36E+07 |
| 70 | Nitrosotenuis sp. | 5.13E+06 | 4.62E+06-5.64E+06 |
| 71 | Oscillibacter sp. | 1.20E+07 | 1.08E+07-1.32E+07 |
| 72 | Chryseobacterium taihuense | 4.96E+06 | 4.46E+06-5.46E+06 |
| 73 | Ruminiclostridium sp. | 1.09E+07 | 9.81E+06-1.20E+07 |
| 74 | Ruminococcus sp. | 4.91E+06 | 4.42E+06-5.40E+06 |
| 75 | Anaerocolumna sp. | 4.70E+06 | 4.23E+06-5.17E+06 |
| 76 | Escherichia coli | 2.08E+06 | 1.87E+06-2.29E+06 |
| 77 | Bacillus sp. | 4.44E+06 | 4.00E+06-4.88E+06 |
| 78 | Amnipila sp. | 1.99E+06 | 1.79E+06-2.19E+06 |
| 79 | Reyranella sp. | 4.27E+06 | 3.84E+06-4.71E+06 |
| 80 | Leuconostoc pseudomesenteroides | 1.92E+06 | 1.73E+06-2.11E+06 |
| 81 | Comamonas terrigena | 4.27E+06 | 3.84E+06-4.71E+06 |
| 82 | Citrobacter amalonaticus | 1.83E+06 | 1.65E+06-2.01E+06 |
| 83 | Enterococcus italicus | 4.27E+06 | 3.84E+06-4.71E+06 |
| 84 | Lactobacillus vaccinostercus | 1.71E+06 | 1.54E+06-1.88E+06 |
| 85 | Butyricicoccus sp. | 4.27E+06 | 3.84E+06-4.71E+06 |
| 86 | Monoglobus sp. | 1.71E+06 | 1.54E+06-1.88E+06 |
| 87 | Kosakonia oryzae | 4.10E+06 | 3.69E+06-4.51E+06 |
| 88 | Micropepsis sp. | 1.71E+06 | 1.54E+06-1.88E+06 |
| 89 | Clostridium tunisiense | 3.70E+06 | 3.33E+06-4.07E+06 |
| 90 | Dechlorosoma sp. | 1.71E+06 | 1.54E+06-1.88E+06 |
| 91 | Ilyobacter delafieldii | 3.61E+06 | 3.25E+06-3.97E+06 |
| 92 | Lacticaseibacillus sp. | 1.54E+06 | 1.39E+06-1.69E+06 |
| 93 | Udaeobacter sp. | 3.42E+06 | 3.08E+06-3.76E+06 |
| 94 | Delftia sp. | 1.54E+06 | 1.39E+06-1.69E+06 |
| 95 | Ruminiclostridium hungatei | 3.10E+06 | 2.79E+06-3.41E+06 |
| 96 | Sporomusa silvacetica | 1.50E+06 | 1.35E+06-1.65E+06 |
| 97 | Nitrosocosmicus oleophilus | 2.85E+06 | 2.57E+06-3.13E+06 |
| 98 | Desulfotomaculum sp. | 1.28E+06 | 1.15E+06-1.41E+06 |
| 99 | Desulfosporomusa polytropa | 2.78E+06 | 2.50E+06-3.06E+06 |
| 100 | Acidipropionibacterium | 1.28E+06 | 1.15E+06-1.41E+06 |
| | acidipropionici | | |
| 101 | Caproiciproducens sp. | 2.78E+06 | 2.50E+06-3.06E+06 |
| 102 | Pandoraea pnomenusa | 1.28E+06 | 1.15E+06-1.41E+06 |
| 103 | Actinomyces sp. | 2.56E+06 | 2.30E+06-2.82E+06 |
| 104 | Herbaspirillum huttiense | 1.28E+06 | 1.15E+06-1.41E+06 |
| 105 | Phenylobacterium sp. | 2.56E+06 | 2.30E+06-2.82E+06 |
| 106 | Papillibacter sp. | 1.28E+06 | 1.15E+06-1.41E+06 |
| 107 | Saccharibacillus sp. | 2.46E+06 | 2.21E+06-2.71E+06 |
| 108 | Pseudomonas otitidis | 1.07E+06 | 9.63E+05-1.18E+06 |
| 109 | Schaalia turicensis | 2.28E+06 | 2.05E+06-2.51E+06 |
| 110 | Desulfosporosinus fructosivorans | 1.04E+06 | 9.36E+05-1.15E+06 |
| 111 | Novosphingobium resinovorum | 2.28E+06 | 2.05E+06-2.51E+06 |
| 112 | Lactobacillus delbrueckii | 1.04E+06 | 9.36E+05-1.15E+06 |
| 113 | Pseudomonas putida | 2.20E+06 | 1.98E+06-2.42E+06 |
| 114 | Anaerocolumna xylanovorans | 9.97E+05 | 8.97E+05-1.10E+06 |
| 115 | Pseudomonas aeruginosa | 2.14E+06 | 1.93E+06-2.35E+06 |
| 116 | Acinetobacter variabilis | 9.97E+05 | 8.97E+05-1.10E+06 |
| 117 | Caulobacter sp. | 2.14E+06 | 1.93E+06-2.35E+06 |
| 118 | Clostridium subterminale | 9.50E+05 | 8.55E+05-1.05E+06 |
| 119 | Acinetobacter ursingii | 8.55E+05 | 7.70E+05-9.40E+05 |
| 120 | Oxobacter sp. | 3.80E+05 | 3.42E+05-4.18E+05 |
| 121 | Acetanaerobacterium sp. | 8.55E+05 | 7.70E+05-9.40E+05 |
| 122 | Clostridium intestinale | 3.80E+05 | 3.42E+05-4.18E+05 |
| 123 | Lactobacillus pentosus | 8.55E+05 | 7.70E+05-9.40E+05 |
| 124 | Sporomusa malonica | 2.85E+05 | 2.57E+05-3.13E+05 |
| 125 | Rhodovastum sp. | 8.55E+05 | 7.70E+05-9.40E+05 |
| 126 | Clostridium homopropionicum | 2.85E+05 | 2.57E+05-3.13E+05 |
| 127 | Paracoccus sp. | 8.55E+05 | 7.70E+05-9.40E+05 |
| 128 | Clostridium malenominatum | 2.85E+05 | 2.57E+05-3.13E+05 |
| 129 | Anaerostignum sp. | 7.33E+05 | 6.60E+05-8.06E+05 |
| 130 | Aeromonas hydrophila | 2.56E+05 | 2.30E+05-2.82E+05 |
| 131 | Desulfosporosinus meridiei | 6.99E+05 | 6.29E+05-7.69E+05 |
| 132 | Acetobacter sp. | 6.84E+05 | 6.16E+05-7.52E+05 |
| 133 | Pseudarthrobacter oxydans | 6.84E+05 | 6.16E+05-7.52E+05 |
| 134 | Azospirillum sp. | 6.65E+05 | 5.98E+05-7.32E+05 |
| 135 | Citrobacter sp. | 6.41E+05 | 5.77E+05-7.05E+05 |
| 136 | Pseudomonas stutzeri | 6.41E+05 | 5.77E+05-7.05E+05 |
| 137 | Herbinix sp. | 6.41E+05 | 5.77E+05-7.05E+05 |
| 138 | Stenotrophomonas sp. | 6.41E+05 | 5.77E+05-7.05E+05 |
| 139 | Serratia marcescens | 6.11E+05 | 5.50E+05-6.72E+05 |
| 140 | Acinetobacter radioresistens | 4.88E+05 | 4.39E+05-5.37E+05 |
| 141 | Desulfosporosinus youngiae | 4.75E+05 | 4.27E+05-5.22E+05 |
| 142 | Desulfofarcimen sp. | 4.27E+05 | 3.84E+05-4.71E+05 |
| 143 | Trabulsiella sp. | 4.27E+05 | 3.84E+05-4.71E+05 |
| 144 | Oxalophagus oxalicus | 4.27E+05 | 3.84E+05-4.71E+05 |
| 145 | Clostridium tyrobutyricum | 7.12E+05 | 6.41E+05-7.83E+05 |
| 146 | Enterococcus sp. | 7.12E+05 | 6.41E+05-7.83E+05 |

In an embodiment, the stabilized consortia include one or more of the following fungal organisms:

TABLE 4
Example Consortium 1-Fungi

| | | | Percentage | | |
|---|---|---|---|---|---|
| # | Genus & Species | Percentage | Range | Copies | Copies Range |
| 1 | Cyberlindnera jadinii | 59.92% | 41.94%-77.90% | 11,731,740 | 8,212,218-15,251,262 |
| 2 | Penicillium herquei | 0.04% | 0.03%-0.05% | 8,230 | 5,761-10,799 |
| 3 | Williopsis sp. | 30.62% | 21.43%-39.81% | 5,995,750 | 4,196,025-7,795,475 |
| 4 | Mortierella hyalina | 0.04% | 0.03%-0.05% | 7,870 | 5,509-10,231 |
| 5 | Pichia kudriavzevii | 6.29% | 4.40%-8.18% | 1,231,570 | 861,099-1,602,041 |
| 6 | Mucor circinelloides | 0.04% | 0.03%-0.05% | 7,870 | 5,509-10,231 |
| 7 | Candida tropicalis | 1.43% | 1.00%-1.86% | 279,090 | 195,363-362,817 |
| 8 | Preussia flanaganii | 0.04% | 0.03%-0.05% | 7,510 | 5,257-9,763 |
| 9 | Mortierella sp. | 0.14% | 0.10%-0.18% | 27,910 | 19,537-36,283 |
| 10 | Penicillium sp. | 0.04% | 0.03%-0.05% | 7,160 | 5,012-9,308 |
| 11 | Penicillium melinii | 0.14% | 0.10%-0.18% | 27,190 | 19,033-35,347 |
| 12 | Mortierella gamsii | 0.04% | 0.03%-0.05% | 7,160 | 5,012-9,308 |
| 13 | Starmera stellimalicola | 0.13% | 0.09%-0.17% | 26,120 | 18,284-33,956 |
| 14 | Purpureocillium | 0.03% | 0.02%-0.04% | 6,080 | 4,256-7,904 |
| | lavendulum | | | | |
| 15 | Mucor irregularis | 0.08% | 0.06%-0.10% | 16,460 | 11,522-21,398 |
| 16 | Monocillium indicum | 0.03% | 0.02%-0.04% | 5,720 | 4,004-7,436 |
| 17 | Penicillium | 0.06% | 0.04%-0.08% | 12,520 | 8,764-16,276 |
| | griseofulvum | | | | |
| 18 | Solicoccozyma aeria | 0.03% | 0.02%-0.04% | 5,720 | 4,004-7,436 |
| 19 | Mortierella alpina | 0.06% | 0.04%-0.08% | 11,450 | 8,015-14,885 |
| 20 | Monocillium mucidum | 0.03% | 0.02%-0.04% | 5,370 | 3,759-6,981 |
| 21 | Mortierella ambigua | 0.06% | 0.04%-0.08% | 11,090 | 7,763-14,417 |
| 22 | Tetracladium sp. | 0.03% | 0.02%-0.04% | 5,370 | 3,759-6,981 |
| 23 | Tausonia pullulans | 0.06% | 0.04%-0.08% | 11,090 | 7,763-14,417 |
| 24 | Trichoderma piluliferum | 0.03% | 0.02%-0.04% | 5,370 | 3,759-6,981 |
| 25 | Stropharia coronilla | 0.06% | 0.04%-0.08% | 11,090 | 7,763-14,417 |
| 26 | Gymnostellatospora | 0.03% | 0.02%-0.04% | 5,010 | 3,507-6,513 |
| | japonica | | | | |
| 27 | Aspergillus caninus | 0.05% | 0.04%-0.06% | 10,020 | 7,014-13,026 |
| 28 | Meyerozyma caribbica | 0.03% | 0.02%-0.04% | 5,010 | 3,507-6,513 |
| 29 | Penicillium bilaiae | 0.05% | 0.04%-0.06% | 10,020 | 7,014-13,026 |
| 30 | Trichoderma harzianum | 0.03% | 0.02%-0.04% | 5,010 | 3,507-6,513 |
| 31 | Pseudogymnoascus | 0.05% | 0.04%-0.06% | 9,300 | 6,510-12,090 |
| | roseus | | | | |
| 32 | Penicillium oxalicum | 0.03% | 0.02%-0.04% | 5,010 | 3,507-6,513 |
| 33 | Talaromyces marne ei | 0.04% | 0.03%-0.05% | 8,230 | 5,761-10,799 |
| 34 | Phaeosphaeriopsis sp. | 0.02% | 0.01%-0.03% | 4,290 | 3,003-5,577 |
| 35 | Penicillium | 0.02% | 0.01%-0.03% | 4,290 | 3,003-5,577 |
| | brevicompactum | | | | |
| 36 | Alternaria longissima | 0.02% | 0.01%-0.03% | 4,290 | 3,003-5,577 |
| 37 | Candida parapsilosis | 0.02% | 0.01%-0.03% | 3,940 | 2,758-5,122 |
| 38 | Pseudostrickeria sp. | 0.02% | 0.01%-0.03% | 3,940 | 2,758-5,122 |
| 39 | Allophoma tropica | 0.02% | 0.01%-0.03% | 3,940 | 2,758-5,122 |
| 40 | Leptosphaeria maculans | 0.02% | 0.01%-0.03% | 3,940 | 2,758-5,122 |
| 41 | Trichoderma pubescens | 0.02% | 0.01%-0.03% | 3,580 | 2,506-4,654 |
| 42 | Cortinarius helvolus | 0.02% | 0.01%-0.03% | 3,580 | 2,506-4,654 |
| 43 | Mortierella sarnyensis | 0.02% | 0.01%-0.03% | 3,580 | 2,506-4,654 |
| 44 | Pyrenochaetopsis | 0.02% | 0.01%-0.03% | 3,580 | 2,506-4,654 |
| | leptospora | | | | |
| 45 | Exophiala sp. | 0.02% | 0.01%-0.03% | 3,580 | 2,506-4,654 |
| 46 | Pseudothielavia terricola | 0.02% | 0.01%-0.03% | 3,580 | 2,506-4,654 |

Alternatively, or additionally, the stabilized consortia may include one or more of bacteria in the following phyla: Proteobacteria, Firmicutes, Bacteroidota, Verrucomicrobiota, Desulfobacterota, or a combination thereof. In particular, when considering the total percentage of bacteria, the consortia may include between about 75% and 85% Proteobacteria, between about 10% and about 15% Bacteroidota, between about 0.5% and about 3% Firmicutes, between about 0.5% and about 2% Verrucomicrobiota, and/or between about 0.5% and about 2% Desulfobacterota.

The stabilized consortia may additionally or alternatively include fungi from the following phyla: Ascomycota, Mortierellomycota, Basidiomycota, Olpidiomycota, or a combination thereof. In particular, when considering the total percentage of fungi, the consortia may include between about 75% and about 85% of Ascomycota, between about 10% and about 15% Mortierellomycota, between about 5% and about 10% Basidiomycota, and/or 1% or less of Olpidiomycota.

In an embodiment, the stabilized consortia include one or more of the following bacterial organisms:

TABLE 5
Example Consortium 2-Bacteria

| # | Genus & Species | Cells/ml | Range Cells/ml |
|---|---|---|---|
| 1 | Tabrizicola sp. | 1.29E+08 | 1.16E+08-1.42E+08 |
| 2 | Pseudomonas putida | 8.31E+06 | 7.48E+06-9.15E+06 |
| 3 | Paracoccus sp. | 5.47E+07 | 4.92E+07-6.01E+07 |
| 4 | Bacteroides sp. | 7.67E+06 | 6.90E+06-8.44E+06 |
| 5 | Acinetobacter junii | 4.35E+07 | 3.92E+07-4.78E+07 |
| 6 | Hydrogenophaga sp. | 7.59E+06 | 6.83E+06-8.35E+06 |
| 7 | Novosphingobium sp. | 3.17E+07 | 2.85E+07-3.49E+07 |
| 8 | Neorhizobium alkalisoli | 7.44E+06 | 6.69E+06-8.19E+06 |
| 9 | Novosphingobium resinovorum | 3.08E+07 | 2.77E+07-3.39E+07 |
| 10 | Methylophilus sp. | 7.36E+06 | 6.62E+06-8.10E+06 |
| 11 | Dysgonomonas sp. | 2.47E+07 | 2.22E+07-2.72E+07 |
| 12 | Desulfobulbus sp. | 7.28E+06 | 6.55E+06-7.99E+06 |
| 13 | Geminisphaera sp. | 2.35E+07 | 2.11E+07-2.59E+07 |
| 14 | Paludibacter sp. | 7.10E+06 | 6.39E+06-7.81E+06 |
| 15 | Pseudomonas sp. | 1.88E+07 | 1.69E+07-2.07E+07 |
| 16 | Solimonas flava | 6.82E+06 | 6.14E+06-7.50E+06 |
| 17 | Flavobacterium sp. | 1.73E+07 | 1.56E+07-1.90E+07 |
| 18 | Pleomorphomonas sp. | 6.03E+06 | 5.43E+06-6.63E+06 |
| 19 | Acinetobacter calcoaceticus | 1.21E+07 | 1.09E+07-1.33E+07 |
| 20 | Sphingobium sp. | 5.85E+06 | 5.27E+06-6.43E+06 |
| 21 | Rhodobacter sp. | 1.18E+07 | 1.06E+07-1.30E+07 |
| 22 | Shewanella dokdonensis | 5.73E+06 | 5.16E+06-6.30E+06 |
| 23 | Klebsiella pneumoniae | 1.05E+07 | 9.45E+06-1.16E+07 |
| 24 | Rhizobium sp. | 5.03E+06 | 4.53E+06-5.53E+06 |
| 25 | Pseudacidovorax intermedius | 1.04E+07 | 9.36E+06-1.15E+07 |
| 26 | Magnetospirillum sp. | 4.95E+06 | 4.45E+06-5.45E+06 |
| 27 | Siphonobacter aquaeclarae | 9.70E+06 | 8.73E+06-1.07E+07 |
| 28 | Oleomonas sagaranensis | 4.80E+06 | 4.32E+06-5.28E+06 |
| 29 | Caulobacter sp. | 8.44E+06 | 7.60E+06-9.28E+06 |
| 30 | Riegeria sp. | 4.27E+06 | 3.84E+06-4.70E+06 |
| 31 | Quatrionicoccus sp. | 4.16E+06 | 3.74E+06-4.58E+06 |
| 32 | Paenirhodobacter sp. | 8.73E+05 | 7.86E+05-9.60E+05 |
| 33 | Ideonella dechloratans | 4.10E+06 | 3.69E+06-4.51E+06 |
| 34 | Roseomonas sp. | 8.65E+05 | 7.79E+05-9.51E+05 |
| 35 | Xanthobacter | 3.95E+06 | 3.55E+06-4.34E+06 |
| | polyaromaticivorans | | |
| 36 | Thiobacillus thioparus | 8.52E+05 | 7.67E+05-9.37E+05 |
| 37 | Ancalomicrobium sp. | 3.78E+06 | 3.40E+06-4.16E+06 |
| 38 | Uliginosibacterium sp. | 8.45E+05 | 7.61E+05-9.29E+05 |
| 39 | Mobilitalea sp. | 3.03E+06 | 2.73E+06-3.33E+06 |
| 40 | Anaerosporobacter sp. | 8.31E+05 | 7.48E+05-9.14E+05 |
| 41 | Pseudomonas oleovorans | 2.99E+06 | 2.69E+06-3.29E+06 |
| 42 | Devosia insulae | 8.31E+05 | 7.48E+05-9.14E+05 |
| 43 | Agrobacterium tumefaciens | 2.97E+06 | 2.67E+06-3.27E+06 |
| 44 | Erythrobacter mathurensis | 8.31E+05 | 7.48E+05-9.14E+05 |
| 45 | Propionivibrio sp. | 2.85E+06 | 2.57E+06-3.13E+06 |
| 46 | Cloacibacterium sp. | 7.69E+05 | 6.92E+05-8.45E+05 |
| 47 | Lacunisphaera sp. | 2.66E+06 | 2.39E+06-2.92E+06 |
| 48 | Chitinophaga pinensis | 7.69E+05 | 6.92E+05-8.45E+05 |
| 49 | Xanthobacter autotrophicus | 2.18E+06 | 1.96E+06-2.40E+06 |
| 50 | Aeromonas hydrophila | 7.69E+05 | 6.92E+05-8.45E+05 |
| 51 | Mucilaginibacter litoreus | 1.97E+06 | 1.77E+06-2.17E+06 |
| 52 | Comamonas aquatica | 7.32E+05 | 6.59E+05-8.05E+05 |
| 53 | Dyadobacter fermentans | 1.62E+06 | 1.46E+06-1.78E+06 |
| 54 | Lactococcus lactis | 7.07E+05 | 6.36E+05-7.78E+05 |
| 55 | Phenylobacterium sp. | 1.62E+06 | 1.46E+06-1.78E+06 |
| 56 | Brevundimonas aurantiaca | 6.86E+05 | 6.17E+05-7.55E+05 |
| 57 | Acinetobacter haemolyticus | 1.61E+06 | 1.45E+06-1.77E+06 |
| 58 | Bosea sp. | 6.44E+05 | 5.80E+05-7.08E+05 |
| 59 | Desulfovibrio sp. | 1.57E+06 | 1.41E+06-1.72E+06 |
| 60 | Clostridium saccharolyticum | 5.61E+05 | 5.05E+05-6.17E+05 |
| 61 | Chitinophaga sancti | 1.50E+06 | 1.35E+06-1.65E+06 |
| 62 | Xanthobacter flavus | 5.20E+05 | 4.68E+05-5.72E+05 |
| 63 | Azospirillum sp. | 1.47E+06 | 1.32E+06-1.61E+06 |
| 64 | Sphingobium yanoikuyae | 4.99E+05 | 4.49E+05-5.49E+05 |
| 65 | Desulfovibrio vulgaris | 1.18E+06 | 1.06E+06-1.30E+06 |
| 66 | Azoarcus olearius | 4.78E+05 | 4.30E+05-5.26E+05 |
| 67 | Catellibacterium terrae | 1.14E+06 | 1.03E+06-1.25E+06 |
| 68 | Rhodovarius lipocyclicus | 4.68E+05 | 4.21E+05-5.15E+05 |
| 69 | Aquaspirillum polymorphum | 1.10E+06 | 9.90E+05-1.21E+06 |
| 70 | Roseomonas rubra | 4.49E+05 | 4.04E+05-4.94E+05 |
| 71 | Starkeya novella | 1.04E+06 | 9.36E+05-1.15E+06 |
| 72 | Paracoccus yeei | 4.43E+05 | 3.99E+05-4.87E+05 |
| 73 | Gracilibacter sp. | 9.64E+05 | 8.68E+05-1.06E+06 |
| 74 | Anaerovorax sp. | 4.26E+05 | 3.83E+05-4.69E+05 |
| 75 | Stenotrophomonas sp. | 4.16E+05 | 3.74E+05-4.58E+05 |
| 76 | Dechlorosoma suillum | 1.87E+05 | 1.68E+05-2.06E+05 |
| 77 | Aeromonas sp. | 4.12E+05 | 3.71E+05-4.53E+05 |
| 78 | Acinetobacter johnsonii | 1.84E+05 | 1.66E+05-2.02E+05 |
| 79 | Anaerotaenia torta | 4.05E+05 | 3.65E+05-4.45E+05 |
| 80 | Parabacteroides chartae | 1.77E+05 | 1.59E+05-1.95E+05 |
| 81 | Pedobacter rhizosphaerae | 3.95E+05 | 3.56E+05-4.34E+05 |
| 82 | Anaerocolumna cellulosilytica | 1.73E+05 | 1.56E+05-1.90E+05 |
| 83 | Rhizobium undicola | 3.88E+05 | 3.49E+05-4.27E+05 |
| 84 | Shinella zoogloeoides | 1.66E+05 | 1.49E+05-1.83E+05 |
| 85 | Citrobacter amalonaticus | 3.86E+05 | 3.47E+05-4.25E+05 |
| 86 | Herbaspirillum huttiense | 1.66E+05 | 1.49E+05-1.83E+05 |
| 87 | Bdellovibrio sp. | 3.74E+05 | 3.37E+05-4.11E+05 |
| 88 | Acinetobacter sp. | 1.52E+05 | 1.37E+05-1.67E+05 |
| 89 | Clostridium beijerinckii | 3.74E+05 | 3.37E+05-4.11E+05 |
| 90 | Propionicimonas sp. | 1.46E+05 | 1.31E+05-1.61E+05 |
| 91 | Lachnotalea sp. | 3.58E+05 | 3.22E+05-3.94E+05 |
| 92 | Bosea thiooxidans | 1.46E+05 | 1.31E+05-1.61E+05 |
| 93 | Variovorax ginsengisoli | 3.12E+05 | 2.81E+05-3.43E+05 |
| 94 | Kaistia hirudinis | 1.46E+05 | 1.31E+05-1.61E+05 |
| 95 | Zoogloea sp. | 2.91E+05 | 2.62E+05-3.20E+05 |
| 96 | Kaistia dalseonensis | 1.46E+05 | 1.31E+05-1.61E+05 |
| 97 | Opitutus sp. | 2.91E+05 | 2.62E+05-3.20E+05 |
| 98 | Kaistia sp. | 1.46E+05 | 1.31E+05-1.61E+05 |
| 99 | Terrimicrobium sacchariphilum | 2.91E+05 | 2.62E+05-3.20E+05 |
| 100 | Mycolicibacterium | 1.46E+05 | 1.31E+05-1.61E+05 |
| | mucogenicum | | |
| 101 | Acinetobacter brisouii | 2.84E+05 | 2.56E+05-3.12E+05 |
| 102 | Asticcacaulis sp. | 1.39E+05 | 1.25E+05-1.53E+05 |
| 103 | Rhizobium gallicum | 2.36E+05 | 2.12E+05-2.60E+05 |
| 104 | Lentilactobacillus sp. | 1.33E+05 | 1.20E+05-1.46E+05 |
| 105 | Comamonas terrigena | 2.24E+05 | 2.02E+05-2.46E+05 |
| 106 | Cellulosimicrobium sp. | 1.25E+05 | 1.12E+05-1.37E+05 |
| 107 | Chthoniobacter sp. | 2.08E+05 | 1.87E+05-2.29E+05 |
| 108 | Pseudomonas stutzeri | 1.25E+05 | 1.12E+05-1.37E+05 |
| 109 | Prosthecobacter debontii | 2.08E+05 | 1.87E+05-2.29E+05 |
| 110 | Mycobacterium gilvum | 1.25E+05 | 1.12E+05-1.37E+05 |
| 111 | Sphingomonas wittichii | 1.87E+05 | 1.68E+05-2.06E+05 |
| 112 | Cereibacter changlensis | 1.25E+05 | 1.12E+05-1.37E+05 |
| 113 | Pseudomonas mendocina | 1.87E+05 | 1.68E+05-2.06E+05 |
| 114 | Variovorax sp. | 1.25E+05 | 1.12E+05-1.37E+05 |
| 115 | Herbinix sp. | 1.87E+05 | 1.68E+05-2.06E+05 |
| 116 | Pseudaeromonas pectinilytica | 1.21E+05 | 1.09E+05-1.33E+05 |
| 117 | Cupriavidus campinensis | 1.87E+05 | 1.68E+05-2.06E+05 |
| 118 | Bacillus circulans | 1.17E+05 | 1.05E+05-1.29E+05 |
| 119 | Fibrisoma sp. | 1.11E+05 | 9.99E+04-1.23E+05 |
| 120 | Moraxella osloensis | 4.16E+04 | 3.74E+04-4.58E+04 |
| 121 | Ensifer adhaerens | 9.98E+04 | 8.98E+04-1.10E+05 |
| 122 | Rhizobium rhizoryzae | 3.33E+04 | 2.99E+04-3.67E+04 |
| 123 | Novosphingobium | 9.70E+04 | 8.73E+04-1.07E+05 |
| | aromaticivorans | | |
| 124 | Clostridium intestinale | 3.23E+04 | 2.91E+04-3.55E+04 |
| 125 | Escherichia coli | 9.50E+04 | 8.55E+04-1.05E+05 |
| 126 | Lactobacillus pentosus | 2.49E+04 | 2.24E+04-2.74E+04 |
| 127 | Shinella fusca | 8.31E+04 | 7.48E+04-9.14E+04 |
| 128 | Roseomonas eburnea | 2.49E+04 | 2.24E+04-2.74E+04 |
| 129 | Roseomonas cervicalis | 8.31E+04 | 7.48E+04-9.14E+04 |
| 130 | Acinetobacter soli | 2.38E+04 | 2.14E+04-2.62E+04 |
| 131 | Rhodoplanes roseus | 8.31E+04 | 7.48E+04-9.14E+04 |
| 132 | Paenibacillus ihuae | 2.08E+04 | 1.87E+04-2.29E+04 |
| 133 | Caulobacter mirabilis | 8.31E+04 | 7.48E+04-9.14E+04 |
| 134 | Enterobacter sp. | 2.08E+04 | 1.87E+04-2.29E+04 |
| 135 | Ruminiclostridium sp. | 7.80E+04 | 7.02E+04-8.58E+04 |
| 136 | Sedimentibacter sp. | 1.78E+04 | 1.60E+04-1.96E+04 |
| 137 | Lactobacillus paracasei | 7.48E+04 | 6.73E+04-8.23E+04 |
| 138 | Flavobacterium akiainvivens | 1.78E+04 | 1.60E+04-1.96E+04 |
| 139 | Pseudomonas alcaligenes | 6.93E+04 | 6.23E+04-7.63E+04 |
| 140 | Paenibacillus graminis | 1.25E+04 | 1.12E+04-1.37E+04 |
| 141 | Comamonas testosteroni | 6.53E+04 | 5.88E+04-7.18E+04 |
| 142 | Azoarcus indigens | 6.24E+04 | 5.62E+04-6.86E+04 |
| 143 | Marinomonas sp. | 5.72E+04 | 5.15E+04-6.29E+04 |
| 144 | Xinfangfangia soli | 5.54E+04 | 4.99E+04-6.09E+04 |
| 145 | Pandoraea pnomenusa | 5.20E+04 | 4.68E+04-5.72E+04 |
| 146 | Imtechium assamiensis | 5.20E+04 | 4.68E+04-5.72E+04 |
| 147 | Raoultella planticola | 4.68E+04 | 4.21E+04-5.15E+04 |
| 148 | Roseomonas aestuarii | 4.16E+04 | 3.74E+04-4.58E+04 |
| 149 | Legionella lytica | 4.16E+04 | 3.74E+04-4.58E+04 |
| 150 | Novosphingobium sediminicola | 4.16E+04 | 3.74E+04-4.58E+04 |
| 151 | Stenotrophomonas maltophilia | 4.16E+04 | 3.74E+04-4.58E+04 |

In an embodiment, the stabilized consortia include one or more of the following fungal organisms:

TABLE 6
Example Consortium 2-Fungi

| | | Per- | Percentage | | Copies |
|---|---|---|---|---|---|
| # | Genus & Species | centage | Range | Copies | Range |
| 1 | Pichia kudriavzevii | 8.7975% | 6%-12% | 56,830 | 49,000-63,000 |
| 2 | Mortierella sp. | 8.7290% | 6%-12% | 56,380 | 48,800-63,300 |
| 3 | Cladosporium | 6.8162% | 5%-8% | 44,030 | 38,300-50,800 |
| | herbarum | | | | |
| 4 | Pseudogymnoascus | 4.6480% | 3%-7% | 30,020 | 26,100-34,600 |
| | roseus | | | | |
| 5 | Cyberlindnera | 3.9377% | 3%-6% | 25,430 | 22,200-28,900 |
| | jadinii | | | | |
| 6 | Trichoderma | 3.6324% | 3%-6% | 23,460 | 20,500-26,400 |
| | asperellum | | | | |
| 7 | Aspergillus | 3.6137% | 3%-6% | 23,340 | 20,360-26,320 |
| | fumigatus | | | | |
| 8 | Talaromyces | 3.4081% | 2%-5% | 22,010 | 19,190-24,810 |
| | marne ei | | | | |
| 9 | Alternaria | 3.0156% | 2%-5% | 19,480 | 17,030-21,980 |
| | eichhorniae | | | | |
| 10 | Penicillium melinii | 2.7850% | 2%-5% | 17,990 | 15,740-20,250 |
| 11 | Solicoccozyma aeria | 2.6542% | 2%-5% | 17,140 | 14,950-19,330 |
| 12 | Penicillium herquei | 2.2492% | 2%-4% | 14,530 | 12,670-16,380 |
| 13 | Penicillium bilaiae | 1.7695% | 1%-3% | 11,430 | 9,970-12,880 |
| 14 | Talaromyces piceae | 1.5826% | 1%-3% | 10,220 | 8,920-11,530 |
| 15 | Penicillium levitum | 1.4517% | 1%-3% | 9,380 | 8,180-10,570 |
| 16 | Mortierella | 1.4455% | 1%-3% | 9,340 | 8,140-10,520 |
| | sarnyensis | | | | |
| 17 | Trichoderma | 1.4268% | 1%-3% | 9,220 | 8,030-10,390 |
| | piluliferum | | | | |
| 18 | Talaromyces sp. | 1.4268% | 1%-3% | 9,220 | 8,030-10,390 |
| 19 | Chrysosporium | 1.2960% | 1%-3% | 8,370 | 7,300-9,430 |
| | lobatum | | | | |
| 20 | Didymella exigua | 1.0592% | 1%-2% | 6,840 | 5,980-7,700 |
| 21 | Aspergillus | 1.0405% | 1%-2% | 6,720 | 5,870-7,570 |
| | chlamydosporus | | | | |
| 22 | Penicillium | 1.0280% | 1%-2% | 6,640 | 5,800-7,480 |
| | roseopurpureum | | | | |
| 23 | Subramaniula | 0.9657% | 1%-2% | 6,240 | 5,450-6,960 |
| | asteroides | | | | |
| 24 | Aspergillus | 0.9595% | 1%-2% | 6,200 | 5,420-6,980 |
| | pseudodeflectus | | | | |
| 25 | Penicillium citrinum | 0.8723% | 1%-2% | 5,630 | 4,930-6,330 |
| 26 | Chrysosporium | 0.8723% | 1%-2% | 5,630 | 4,930-6,330 |
| | pseudomerdarium | | | | |
| 27 | Mortierella | 0.8660% | 1%-2% | 5,590 | 4,880-6,300 |
| | rishikesha | | | | |
| 28 | Neobulgaria sp. | 0.8474% | 1%-2% | 5,470 | 4,780-6,160 |
| 29 | Curvularia | 0.8349% | 1%-2% | 5,390 | 4,700-6,080 |
| | verruculosa | | | | |
| 30 | Clavaria sp. | 0.7975% | 1%-2% | 5,150 | 4,490-5,810 |
| 31 | Plectosphaerella | 0.7601% | 1%-2% | 4,910 | 4,290-5,530 |
| | cucumerina | | | | |
| 32 | Mortierella | 0.7539% | 1%-2% | 4,870 | 4,240-5,500 |
| | minutissima | | | | |
| 33 | Williopsis sp. | 0.7227% | 1%-2% | 4,670 | 4,080-5,260 |
| 34 | Penicillium | 0.7103% | 1%-2% | 4,590 | 4,010-5,170 |
| | parviverrucosum | | | | |
| 35 | Panaeolus fimicola | 0.6978% | 1%-2% | 4,510 | 3,940-5,090 |
| 36 | Tausonia pullulans | 0.6916% | 1%-2% | 4,470 | 3,910-5,050 |
| 37 | Psathyrella ichnusae | 0.6854% | 1%-2% | 4,430 | 3,870-5,010 |
| 38 | Arachnomyces | 0.6542% | 1%-2% | 4,230 | 3,690-4,770 |
| | gracilis | | | | |
| 39 | Solicoccozyma | 0.6355% | 1%-2% | 4,100 | 3,580-4,620 |
| | terrea | | | | |
| 40 | Mortierella | 0.6231% | 1%-2% | 4,020 | 3,510-4,530 |
| | antarctica | | | | |
| 41 | Coniothyrium | 0.5919% | 1%-2% | 3,820 | 3,330-4,310 |
| | cerealis | | | | |
| 42 | Neodactylaria sp. | 0.5857% | 1%-2% | 3,780 | 3,300-4,260 |
| 43 | Penicillium | 0.5607% | 1%-2% | 3,620 | 3,160-4,080 |
| | pimiteouiense | | | | |
| 44 | Penicillium | 0.5607% | 1%-2% | 3,620 | 3,160-4,080 |
| | simplicissimum | | | | |
| 45 | Humicola olivacea | 0.5171% | 1%-2% | 3,340 | 2,910-3,770 |
| 46 | Arachnomyces kanei | 0.4860% | 0.001%-1% | 3,140 | 2,740-3,540 |
| 47 | Mortierella lignicola | 0.4611% | 0.001%-1% | 2,980 | 2,590-3,370 |
| 48 | Thelebolus globosus | 0.4548% | 0.001%-1% | 2,940 | 2,560-3,320 |
| 49 | Bipolaris | 0.4424% | 0.001%-1% | 2,860 | 2,490-3,270 |
| | microlaenae | | | | |
| 50 | Penicillium | 0.4361% | 0.001%-1% | 2,820 | 2,460-3,180 |
| | erubescens | | | | |
| 51 | Polyschema | 0.4237% | 0.001%-1% | 2,740 | 2,390-3,090 |
| | sclerotigenum | | | | |
| 52 | Phaeosphaeria sp. | 0.4174% | 0.001%-1% | 2,700 | 2,360-3,040 |
| 53 | Sarocladium | 0.4112% | 0.001%-1% | 2,660 | 2,320-3,000 |
| | strictum | | | | |
| 54 | Penicillium | 0.4112% | 0.001%-1% | 2,660 | 2,320-3,000 |
| | restrictum | | | | |
| 55 | Fusarium | 0.3925% | 0.001%-1% | 2,540 | 2,210-2,870 |
| | proliferatum | | | | |
| 56 | Penicillium | 0.3863% | 0.001%-1% | 2,500 | 2,180-2,820 |
| | argentinense | | | | |
| 57 | Alternaria | 0.3801% | 0.001%-1% | 2,450 | 2,140-2,760 |
| | metachromatica | | | | |
| 58 | Coniothyrium sp. | 0.3801% | 0.001%-1% | 2,450 | 2,140-2,760 |
| 59 | Penicillium sp. | 0.3801% | 0.001%-1% | 2,450 | 2,140-2,760 |
| 60 | Talaromyces | 0.3738% | 0.001%-1% | 2,410 | 2,100-2,720 |
| | purpureogenus | | | | |
| 61 | Aspergillus niger | 0.3738% | 0.001%-1% | 2,410 | 2,100-2,720 |
| 62 | Mortierella gamsii | 0.3676% | 0.001%-1% | 2,370 | 2070-2,680 |
| 63 | Lachnum sp. | 0.3676% | 0.001%-1% | 2,370 | 2,070-2,680 |
| 64 | Gymnopus sp. | 0.3614% | 0.001%-1% | 2,330 | 2,030-2,640 |
| 65 | Penicillium rubens | 0.3489% | 0.001%-1% | 2,250 | 1,960-2,540 |
| 66 | Alternaria photistica | 0.3240% | 0.001%-1% | 2,090 | 1,820-2,360 |
| 67 | Sporormiella | 0.3115% | 0.001%-1% | 2,010 | 1,750-2,270 |
| | megalospora | | | | |
| 68 | Didymella pinodes | 0.2928% | 0.001%-1% | 1,890 | 1,650-2,130 |
| 69 | Phallus rugulosus | 0.2928% | 0.001%-1% | 1,890 | 1,650-2,130 |
| 70 | Alternaria | 0.2928% | 0.001%-1% | 1,890 | 1,650-2,130 |
| | longissima | | | | |
| 71 | Coniochaeta canina | 0.2928% | 0.001%-1% | 1,890 | 1,650-2,130 |
| 72 | Cutaneotrichosporon | 0.2928% | 0.001%-1% | 1,890 | 1,650-2,130 |
| | jirovecii | | | | |
| 73 | Aspergillus | 0.2804% | 0.001%-1% | 1,810 | 1,580-2,040 |
| | ochraceus | | | | |
| 74 | Monocillium | 0.2741% | 0.001%-1% | 1,770 | 1,540-1,990 |
| | mucidum | | | | |
| 75 | Penicillium | 0.2741% | 0.001%-1% | 1,770 | 1,540-1,990 |
| | catenatum | | | | |
| 76 | Microscypha sp. | 0.2679% | 0.001%-1% | 1,730 | 1,510-1,950 |
| 77 | Sebacina sp. | 0.2679% | 0.001%-1% | 1,730 | 1,510-1,950 |
| 78 | Talaromyces stollii | 0.2492% | 0.001%-1% | 1,610 | 1,400-1,820 |
| 79 | Oidiodendron | 0.2492% | 0.001%-1% | 1,610 | 1,400-1,820 |
| | cereale | | | | |
| 81 | Hannaella oryzae | 0.2430% | 0.001%-1% | 1,570 | 1,370-1,780 |
| 82 | Furcasterigmium | 0.2430% | 0.001%-1% | 1,570 | 1,370-1,780 |
| | furcatum | | | | |
| 83 | Fusarium solani | 0.2305% | 0.001%-1% | 1,490 | 1,300-1,680 |
| 84 | Clonostachys rosea | 0.2243% | 0.001%-1% | 1,450 | 1,270-1,640 |
| 85 | Aspergillus wentii | 0.2181% | 0.001%-1% | 1,410 | 1,230-1,600 |
| 86 | Kernia pachypleura | 0.2118% | 0.001%-1% | 1,370 | 1,190-1,560 |
| 87 | Olpidium brassicae | 0.2118% | 0.001%-1% | 1,370 | 1,190-1,560 |
| 88 | Penicillium steckii | 0.1807% | 0.001%-1% | 1,170 | 1,020-1,320 |
| 89 | Byssochlamys | 0.1807% | 0.001%-1% | 1,170 | 1,020-1,320 |
| | lagunculariae | | | | |
| 90 | Acrocalymma vagum | 0.1682% | 0.001%-1% | 1,090 | 950-1,240 |
| 91 | Mortierella alpina | 0.1620% | 0.001%-1% | 1,050 | 910-1,190 |
| 92 | Trichoderma | 0.1620% | 0.001%-1% | 1,050 | 910-1,190 |
| | harzianum | | | | |
| 93 | Cephalotrichum | 0.1495% | 0.001%-1% | 970 | 840-1,110 |
| | microsporum | | | | |
| 94 | Stemphylium | 0.1308% | 0.001%-1% | 850 | 740-960 |
| | vesicarium | | | | |
| 95 | Leohumicola | 0.1184% | 0.001%-1% | 760 | 660-860 |
| | verrucosa | | | | |
| 96 | Aspergillus | 0.0935% | 0.001%-1% | 600 | 520-680 |
| | subversicolor | | | | |
| 97 | Wallemia sebi | 0.0810% | 0.001%-1% | 520 | 450-590 |
| 98 | Trichosporon asahii | 0.0810% | 0.001%-1% | 520 | 450-590 |
| 99 | Rhizoctonia solani | 0.0623% | 0.001%-1% | 400 | 350-450 |

Alternatively or additionally, the stabilized consortia may include one or more of bacteria in the following phyla: Proteobacteria, Firmicutes, Bacteroidota, Desulfobacterota, or a combination thereof. In particular, when considering the total percentage of bacteria, the consortia may include between about 92% and 98% Proteobacteria, between about 1% and about 5% Firmicutes, about 1% or less Bacteroidota, and/or about 1% or less Desulfobacterota.

The stabilized consortia may additionally or alternatively include fungi from the following phyla: Ascomycota, Basidiomycota, Mortierellomycota, Mucoromycota, or a combination thereof. In particular, when considering the total percentage of fungi, the consortia may include between about 75% and about 85% of Ascomycota, between about 5% and about 15% Basidiomycota, between about 1% and about 8% Mortierellomycota, and/or between about 1% and about 8% of Mucoromycota.

In an embodiment, the stabilized consortia include one or more of the following bacterial organisms:

TABLE 7
Example Consortium 3-Bacteria

| # | Genus & Species | Cells/mL | Range Cells/mL |
|---|---|---|---|
| 1 | Novosphingobium resinovorum | 2.81E+08 | 2.53E+08 to 3.09E+08 |
| 2 | Citrobacter amalonaticus | 1.30E+07 | 1.17E+07 to 1.43E+07 |
| 3 | Klebsiella pneumoniae | 1.74E+08 | 1.57E+08 to 1.91E+08 |
| 4 | Yersinia pestis | 1.08E+07 | 9.72E+06 to 1.19E+07 |
| 5 | Acinetobacter calcoaceticus | 8.54E+07 | 7.69E+07 to 9.39E+07 |
| 6 | Pleomorphomonas sp. | 1.01E+07 | 9.09E+06 to 1.11E+07 |
| 7 | Pseudomonas sp. | 6.28E+07 | 5.65E+07 to 6.91E+07 |
| 8 | Caulobacter sp. | 7.86E+06 | 7.07E+06 to 8.65E+06 |
| 9 | Acinetobacter haemolyticus | 3.43E+07 | 3.09E+07 to 3.77E+07 |
| 10 | Tabrizicola sp. | 7.46E+06 | 6.71E+06 to 8.21E+06 |
| 11 | Pseudomonas oleovorans | 3.11E+07 | 2.80E+07 to 3.42E+07 |
| 12 | Kosakonia oryzae | 5.78E+06 | 5.20E+06 to 6.36E+06 |
| 13 | Zoogloea sp. | 2.61E+07 | 2.35E+07 to 2.87E+07 |
| 14 | Prevotella paludivivens | 5.14E+06 | 4.63E+06 to 5.65E+06 |
| 15 | Pseudomonas putida | 2.50E+07 | 2.25E+07 to 2.75E+07 |
| 16 | Shewanella dokdonensis | 4.96E+06 | 4.46E+06 to 5.46E+06 |
| 17 | Neorhizobium alkalisoli | 2.48E+07 | 2.23E+07 to 2.73E+07 |
| 18 | Aeromonas hydrophila | 4.76E+06 | 4.29E+06 to 5.23E+06 |
| 19 | Rhizobium sp. | 2.29E+07 | 2.06E+07 to 2.52E+07 |
| 20 | Clostridium beijerinckii | 4.52E+06 | 4.07E+06 to 4.57E+06 |
| 21 | Riegeria sp. | 2.04E+07 | 1.84E+07 to 2.24E+07 |
| 22 | Herbaspirillum huttiense | 3.68E+06 | 3.31E+06 to 4.04E+06 |
| 23 | Rhodobacter sp. | 1.89E+07 | 1.70E+07 to 2.08E+07 |
| 24 | Lactococcus lactis | 3.38E+06 | 3.04E+06 to 3.71E+06 |
| 25 | Acinetobacter junii | 1.88E+07 | 1.69E+07 to 2.07E+07 |
| 26 | Acinetobacter brisouii | 2.84E+06 | 2.56E+06 to 3.12E+06 |
| 27 | Agrobacterium tumefaciens | 1.69E+07 | 1.52E+07 to 1.86E+07 |
| 28 | Paracoccus sp. | 2.82E+06 | 2.54E+06 to 3.10E+06 |
| 29 | Stenotrophomonas sp. | 1.46E+07 | 1.31E+07 to 1.60E+07 |
| 30 | Acinetobacter johnsonii | 2.66E+06 | 2.39E+06 to 2.92E+06 |
| 31 | Pseudomonas mendocina | 2.65E+06 | 2.38E+06 to 2.91E+06 |
| 32 | Aeromonas sp. | 8.26E+05 | 7.43E+05 to 9.09E+05 |
| 33 | Brevundimonas aurantiaca | 2.52E+06 | 2.27E+06 to 2.77E+06 |
| 34 | Sphingobium yanoikuyae | 7.56E+05 | 6.80E+05 to 8.32E+05 |
| 35 | Ideonella dechloratans | 2.05E+06 | 1.85E+06 to 2.26E+06 |
| 36 | Novosphingobium sp. | 7.05E+05 | 6.35E+05 to 7.75E+05 |
| 37 | Raoultella ornithinolytica | 1.93E+06 | 1.73E+06 to 2.12E+06 |
| 38 | Desulfovibrio sp. | 7.05E+05 | 6.35E+05 to 7.75E+05 |
| 39 | Paenirhodobacter sp. | 1.91E+06 | 1.72E+06 to 2.10E+06 |
| 40 | Pseudomonas alcaligenes | 6.38E+05 | 5.74E+05 to 7.02E+05 |
| 41 | Dysgonomonas sp. | 1.81E+06 | 1.63E+06 to 1.99E+06 |
| 42 | Rhizobium gallicum | 5.71E+05 | 5.14E+05 to 6.28E+05 |
| 43 | Propionivibrio sp. | 1.71E+06 | 1.54E+06 to 1.88E+06 |
| 44 | Desulfosporomusa polytropa | 5.37E+05 | 4.83E+05 to 5.91E+05 |
| 45 | Ancalomicrobium sp. | 1.66E+06 | 1.49E+06 to 1.82E+06 |
| 46 | Shinella zoogloeoides | 5.04E+05 | 4.54E+05 to 5.54E+05 |
| 47 | Methylophilus sp. | 1.36E+06 | 1.22E+06 to 1.49E+06 |
| 48 | Marinomonas sp. | 5.04E+05 | 4.54E+05 to 5.54E+05 |
| 49 | Comamonas aquatica | 1.33E+06 | 1.20E+06 to 1.46E+06 |
| 50 | Bacteroides luti | 4.84E+05 | 4.36E+05 to 5.32E+05 |
| 51 | Escherichia coli | 1.27E+06 | 1.14E+06 to 1.40E+06 |
| 52 | Flavobacterium sp. | 4.61E+05 | 4.15E+05 to 5.07E+05 |
| 53 | Acinetobacter sp. | 1.21E+06 | 1.09E+06 to 1.32E+06 |
| 54 | Enterobacter hormaechei | 4.41E+05 | 3.97E+05 to 4.85E+05 |
| 55 | Paracoccus yeei | 1.18E+06 | 1.06E+06 to 1.30E+06 |
| 56 | Moraxella osloensis | 4.03E+05 | 3.63E+05 to 4.43E+05 |
| 57 | Anaerocolumna sp. | 1.06E+06 | 9.54E+05 to 1.17E+06 |
| 58 | Raoultella planticola | 3.78E+05 | 3.40E+05 to 4.16E+05 |
| 59 | Sporomusa sp. | 1.03E+06 | 9.27E+05 to 1.13E+06 |
| 60 | Chitinophaga sancti | 3.69E+05 | 3.32E+05 to 4.06E+05 |
| 61 | Roseomonas sp. | 1.03E+06 | 9.27E+05 to 1.13E+06 |
| 62 | Mycoplana sp. | 3.36E+05 | 3.02E+05 to 3.70E+05 |
| 63 | Lactobacillus pentosus | 1.03E+06 | 9.27E+05 to 1.13E+06 |
| 64 | Azospirillum sp. | 2.91E+05 | 2.62E+05 to 3.21E+05 |
| 65 | Pseudacidovorax intermedius | 1.01E+06 | 9.09E+05 to 1.11E+06 |
| 66 | Acinetobacter variabilis | 2.86E+05 | 2.57E+05 to 3.15E+05 |
| 67 | Siphonobacter aquaeclarae | 8.73E+05 | 7.86E+05 to 9.60E+05 |
| 68 | Pseudomonas nitroreducens | 2.82E+05 | 2.54E+05 to 3.09E+05 |
| 69 | Anaerotaenia torta | 8.57E+05 | 7.71E+05 to 9.43E+05 |
| 70 | Catellibacterium terrae | 2.52E+05 | 2.27E+05 to 2.77E+05 |
| 71 | Bacillus circulans | 8.43E+05 | 7.59E+05 to 9.27E+05 |
| 72 | Lentilactobacillus sp. | 2.42E+05 | 2.18E+05 to 2.66E+05 |
| 73 | Rhizobium petrolearium | 8.40E+05 | 7.56E+05 to 9.24E+05 |
| 74 | Shinella kummerowiae | 2.35E+05 | 2.12E+05 to 2.59E+05 |
| 75 | Propionispora vibrioides | 2.27E+05 | 2.04E+05 to 2.49E+05 |
| 76 | Enterobacter cancerogenus | 8.82E+04 | 7.94E+04 to 9.70E+04 |
| 77 | Ochrobactrum intermedium | 2.02E+05 | 1.82E+05 to 2.22E+05 |
| 78 | Klebsiella sp. | 8.82E+04 | 7.94E+04 to 9.70E+04 |
| 79 | Comamonas terrigena | 2.02E+05 | 1.82E+05 to 2.22E+05 |
| 80 | Acinetobacter tandoii | 8.40E+04 | 7.56E+04 to 8.68E+04 |
| 81 | Oleomonas sagaranensis | 2.02E+05 | 1.82E+05 to 2.22E+05 |
| 82 | Azotobacter chroococcum | 8.40E+04 | 7.56E+04 to 8.68E+04 |
| 83 | Cloacibacterium haliotis | 2.02E+05 | 1.82E+05 to 2.22E+05 |
| 84 | Desulfovibrio vulgaris | 8.06E+04 | 7.25E+04 to 8.87E+04 |
| 85 | Xanthobacter | 2.02E+05 | 1.82E+05 to 2.22E+05 |
| | polyaromaticivorans | | |
| 86 | Dyadobacter fermentans | 7.56E+04 | 6.80E+04 to 8.32E+04 |
| 87 | Lactiplantibacillus sp. | 1.81E+05 | 1.63E+05 to 1.99E+05 |
| 88 | Pedobacter rhizosphaerae | 7.56E+04 | 6.80E+04 to 8.32E+04 |
| 89 | Paenirhodobacter enshiensis | 1.51E+05 | 1.36E+05 to 1.66E+05 |
| 90 | Enterobacter sp. | 7.56E+04 | 6.80E+04 to 8.32E+04 |
| 91 | Hydrogenophaga sp. | 1.51E+05 | 1.36E+05 to 1.66E+05 |
| 92 | Yersinia bercovieri | 7.20E+04 | 6.48E+04 to 7.92E+04 |
| 93 | Pelosinus sp. | 1.46E+05 | 1.31E+05 to 1.61E+05 |
| 94 | Acinetobacter lwoffii | 7.20E+04 | 6.48E+04 to 7.92E+04 |
| 95 | Anaerospora sp. | 1.39E+05 | 1.25E+05 to 1.53E+05 |
| 96 | Lactobacillus coryniformis | 6.05E+04 | 5.45E+04 to 6.65E+04 |
| 97 | Shinella fusca | 1.34E+05 | 1.21E+05 to 1.47E+05 |
| 98 | Lactobacillus perolens | 6.05E+04 | 5.45E+04 to 6.65E+04 |
| 99 | Acinetobacter soli | 1.30E+05 | 1.17E+05 to 1.43E+05 |
| 100 | Ensifer adhaerens | 6.05E+04 | 5.45E+04 to 6.65E+04 |
| 101 | Azoarcus olearius | 1.26E+05 | 1.13E+05 to 1.39E+05 |
| 102 | Pseudomonas psychrotolerans | 6.05E+04 | 5.45E+04 to 6.65E+04 |
| 103 | Lactobacillus vaccinostercus | 1.21E+05 | 1.09E+05 to 1.32E+05 |
| 104 | Comamonas testosteroni | 5.76E+04 | 5.18E+04 to 6.34E+04 |
| 105 | Agrobacterium albertimagni | 1.01E+05 | 9.09E+04 to 1.11E+05 |
| 106 | Sporomusa malonica | 5.04E+04 | 4.54E+04 to 5.54E+04 |
| 107 | Asticcacaulis sp. | 1.01E+05 | 9.09E+04 to 1.11E+05 |
| 108 | Sphingobium sp. | 1.01E+05 | 9.09E+04 to 1.11E+05 |
| 109 | Lactobacillus paracasei | 1.01E+05 | 9.09E+04 to 1.11E+05 |
| 110 | Anaerosporobacter sp. | 1.01E+05 | 9.09E+04 to 1.11E+05 |
| 111 | Acinetobacter venetianus | 1.01E+05 | 9.09E+04 to 1.11E+05 |
| 112 | Pseudomonas stutzeri | 1.01E+05 | 9.09E+04 to 1.11E+05 |

In an embodiment, the stabilized consortia include one or more of the following fungal organisms:

TABLE 7
Example Consortium 3-Fungi

| | | | Percentage | | |
|---|---|---|---|---|---|
| # | Genus & Species | Percentage | Range | Count | Count Range |
| 1 | Cyberlindnera jadinii | 24.26% | 31.54%-16.98% | 155,630 | 201,921-109,339 |
| 2 | Actinomucor elegans | 1.21% | 1.57%-0.85% | 7,740 | 10,062-5,418 |
| 3 | Aspergillus fumigatus | 5.12% | 6.66%-3.58% | 32,850 | 42,705-22,995 |
| 4 | Penicillium levitum | 1.13% | 1.47%-0.79% | 7,250 | 9,425-5,075 |
| 5 | Penicillium bilaiae | 5.11% | 6.64%-3.58% | 32,810 | 42,653-22,467 |
| 6 | Fusarium solani | 1.12% | 1.46%-0.79% | 7,200 | 9,360-5,040 |
| 7 | Penicillium melinii | 5.05% | 6.57%-3.53% | 32,400 | 42,120-22,080 |
| 8 | Mortierella alpina | 1.10% | 1.43%-0.77% | 7,070 | 9,201-4,939 |
| 9 | Scedosporium dehoogii | 3.26% | 4.24%-2.28% | 20,880 | 27,144-14,016 |
| 10 | Penicillium restrictum | 1.09% | 1.42%-0.76% | 7,020 | 9,126-4,914 |
| 11 | Mucor circinelloides | 3.02% | 3.93%-2.11% | 19,350 | 25,155-13,545 |
| 12 | Didymella exigua | 1.05% | 1.36%-0.73% | 6,710 | 8,723-4,697 |
| 13 | Alternaria eichhorniae | 2.50% | 3.25%-1.75% | 16,020 | 20,826-11,214 |
| 14 | Scedosporium boydii | 0.96% | 1.25%-0.67% | 6,170 | 8,021-4,319 |
| 15 | Mortierella rishikesha | 2.13% | 2.77%-1.49% | 13,680 | 17,784-9,576 |
| 16 | Exophiala sp. | 0.93% | 1.21%-0.65% | 5,990 | 7,787-4,193 |
| 17 | Solicoccozyma aeria | 2.10% | 2.73%-1.47% | 13,460 | 17,498-9,422 |
| 18 | Aspergillus caninus | 0.93% | 1.21%-0.65% | 5,940 | 7,722-4,158 |
| 19 | Williopsis sp. | 2.04% | 2.65%-1.42% | 13,100 | 17,030-9,070 |
| 20 | Gibellulopsis serrae | 0.91% | 1.18%-0.63% | 5,810 | 7,553-4,067 |
| 21 | Penicillium | 1.84% | 2.39%-1.29% | 11,790 | 15,347-8,233 |
| | simplicissimum | | | | |
| 22 | Vishniacozyma victoriae | 0.91% | 1.18%-0.63% | 5,810 | 7,553-4,067 |
| 23 | Solicoccozyma terrea | 1.82% | 2.37%-1.27% | 11,700 | 15,210-8,190 |
| 24 | Penicillium herquei | 0.89% | 1.16%-0.62% | 5,720 | 7,436-4,004 |
| 25 | Penicillium argentinense | 1.61% | 2.09%-1.12% | 10,350 | 13,455-7,245 |
| 26 | Cunninghamella echinulata | 0.88% | 1.15%-0.62% | 5,630 | 7,319-3,941 |
| 27 | Tausonia pullulans | 1.59% | 2.07%-1.11% | 10,170 | 13,221-7,119 |
| 28 | Chaetosphaeronema sp. | 0.87% | 1.13%-0.60% | 5,580 | 7,254-3,906 |
| 29 | Mortierella minutissima | 1.52% | 1.98%-1.06% | 9,720 | 12,666-6,798 |
| 30 | Chrysosporium sp. | 0.84% | 1.09%-0.58% | 5,400 | 7,020-3,780 |
| 31 | Pichia kudriavzevii | 1.52% | 1.98%-1.06% | 9,720 | 12,666-6,798 |
| 32 | Filobasidium magnum | 0.82% | 1.07%-0.57% | 5,270 | 6,851-3,689 |
| 33 | Pseudogymnoascus roseus | 1.51% | 1.96%-1.05% | 9,680 | 12,544-6,816 |
| 34 | Phallus rugulosus | 0.81% | 1.05%-0.56% | 5,180 | 6,734-3,626 |
| 35 | Epicoccum thailandicum | 0.79% | 1.03%-0.55% | 5,090 | 6,617-3,563 |
| 36 | Aspergillus sp. | 0.79% | 1.03%-0.55% | 5,040 | 6,552-3,528 |
| 37 | Cladosporium herbarum | 0.74% | 0.96%-0.51% | 4,770 | 6,201-3,339 |
| 38 | Oidiodendron echinulatum | 0.71% | 0.92%-0.49% | 4,550 | 5,915-3,185 |
| 39 | Penicillium polonicum | 0.69% | 0.90%-0.48% | 4,460 | 5,798-3,122 |
| 40 | Plectosphaerella | 0.67% | 0.87%-0.46% | 4,280 | 5,564-2,996 |
| | cucumerina | | | | |
| 41 | Oedocephalum nayoroense | 0.62% | 0.81%-0.43% | 3,960 | 5,148-2,772 |
| 42 | Mortierella gamsii | 0.60% | 0.78%-0.42% | 3,870 | 5,036-2,704 |
| 43 | Penicillium griseofulvum | 0.60% | 0.78%-0.42% | 3,870 | 5,036-2,704 |
| 44 | Penicillium | 0.56% | 0.73%-0.39% | 3,600 | 4,680-2,520 |
| | parviverrucosum | | | | |
| 45 | Fusarium oxysporum | 0.56% | 0.73%-0.39% | 3,600 | 4,680-2,520 |
| 46 | Hirsutella minnesotensis | 0.55% | 0.72%-0.38% | 3,560 | 4,628-2,492 |
| 47 | Aspergillus flavus | 0.54% | 0.70%-0.37% | 3,470 | 4,511-2,429 |
| 48 | Penicillium citrinum | 0.53% | 0.69%-0.37% | 3,420 | 4,446-2,394 |
| 49 | Penicillium atrovenetum | 0.52% | 0.68%-0.35% | 3,330 | 4,329-2,331 |
| 50 | Chrysosporium | 0.51% | 0.66%-0.35% | 3,240 | 4,212-2,268 |
| | synchronum | | | | |
| 51 | Oidiodendron cereale | 0.48% | 0.62%-0.33% | 3,110 | 4,043-2,177 |
| 52 | Hymenoscyphus menthae | 0.48% | 0.62%-0.33% | 3,110 | 4,043-2,177 |
| 53 | Talaromyces marne ei | 0.47% | 0.61%-0.32% | 3,020 | 3,926-2,114 |
| 54 | Aspergillus ochraceus | 0.38% | 0.49%-0.26% | 2,430 | 3,159-1,701 |
| 55 | Talaromyces radicus | 0.36% | 0.47%-0.25% | 2,300 | 2,990-1,610 |
| 56 | Mortierella elongata | 0.35% | 0.46%-0.24% | 2,250 | 2,925-1,575 |
| 57 | Penicillium sp. | 0.31% | 0.40%-0.21% | 1,980 | 2,574-1,386 |
| 58 | Microascus trigonosporus | 0.29% | 0.38%-0.20% | 1,890 | 2,457-1,323 |
| 59 | Humicola fuscoatra | 0.29% | 0.38%-0.20% | 1,890 | 2,457-1,323 |
| 60 | Vishniacozyma tephrensis | 0.29% | 0.38%-0.20% | 1,850 | 2,405-1,295 |
| 61 | Aspergillus terreus | 0.29% | 0.38%-0.20% | 1,850 | 2,405-1,295 |
| 62 | Neosetophoma samarorum | 0.28% | 0.36%-0.19% | 1,800 | 2,340-1,260 |
| 63 | Aureobasidium pullulans | 0.26% | 0.34%-0.18% | 1,670 | 2,171-1,169 |
| 64 | Wallemia canadensis | 0.25% | 0.33%-0.18% | 1,620 | 2,106-1,134 |
| 65 | Penicillium | 0.25% | 0.33%-0.18% | 1,580 | 2,054-1,106 |
| | brevicompactum | | | | |
| 66 | Circinella muscae | 0.25% | 0.33%-0.18% | 1,580 | 2,054-1,106 |
| 67 | Botrytis caroliniana | 0.24% | 0.31%-0.17% | 1,530 | 1,989-1,071 |
| 68 | Aspergillus penicillioides | 0.24% | 0.31%-0.17% | 1,530 | 1,989-1,071 |
| 69 | Aspergillus rugulosus | 0.22% | 0.29%-0.15% | 1,440 | 1,872-1,008 |
| 70 | Pyrenochaetopsis | 0.22% | 0.29%-0.15% | 1,400 | 1,820-980 |
| | leptospora | | | | |
| 71 | Mortierella sarnyensis | 0.20% | 0.26%-0.14% | 1,310 | 1,703-917 |
| 72 | Preussia sp. | 0.19% | 0.25%-0.13% | 1,220 | 1,586-854 |
| 73 | Malbranchea flocciformis | 0.18% | 0.24%-0.13% | 1,130 | 1,469-791 |
| 74 | Malbranchea cinnamomea | 0.16% | 0.21%-0.11% | 1,040 | 1,352-728 |
| 75 | Microdochium sp. | 0.15% | 0.20%-0.11% | 950 | 1,235-665 |
| 76 | Fusarium pseudensiforme | 0.14% | 0.19%-0.10% | 900 | 1,170-630 |
| 77 | Chaetomium acropullum | 0.12% | 0.16%-0.09% | 770 | 1,001-539 |
| 78 | Humicola olivacea | 0.09% | 0.12%-0.07% | 590 | 767-413 |
| 79 | Vishniacozyma globispora | 0.08% | 0.11%-0.06% | 500 | 650-350 |
| 80 | Candida tropicalis | 0.08% | 0.11%-0.06% | 500 | 650-350 |

The identity of the microorganisms used in the consortia described herein was ascertained using a microbiome analysis. The microbiome analysis utilized next-gen sequencing in combination with the addition of a known quantity of an exogenous spike-in, which enables the knowledge of the total microbial load in a sample. After the sequencing and data processing, the relative abundance of the exogenous spike-in was used to extrapolate the original absolute quantity of the 16S copies of the sample species, while knowledge of the number of gene copies per genome in the species was used to calculate the number of cells. Specifically, the sequencing and analysis was conducted using commercially-available techniques using the platform GHEOM® by Biome Makers.

Further discussion of methods of identifying and characterizing microbial consortia can be found in U.S. patent application Ser. Nos. 17/703,095, 17/665,332, 17/119,972, 17/587,046, and 62/263,488 each of which are herein incorporated by reference in their entirety.

Example Consortium 1 was deposited with the Patent Depository of the National Center for Agricultural Utilization Research Agricultural Research Service, U.S. Department of Agriculture, 1815 North University Street, Peoria, Illinois 61604 U.S.A. and assigned NRRL No. XXXXX. Example Consortium 2 was deposited with the Patent Depository of the National Center for Agricultural Utilization Research Agricultural Research Service, U.S. Department of Agriculture, 1815 North University Street, Peoria, Illinois 61604 U.S.A. and assigned NRRL No. YYYYY. Example Consortium 3 was deposited with the Patent Depository of the National Center for Agricultural Utilization Research Agricultural Research Service, U.S. Department of Agriculture, 1815 North University Street, Peoria, Illinois 61604 U.S.A. and assigned NRRL No. ZZZZZ. Each of these deposits will be maintained under the terms of the Budapest Treaty on the International Recognition of the Deposit of Microorganisms for the Purposes of Patent Procedure. This deposit was made merely as a convenience for those of skill in the art and are not an admission that a deposit is required under 35 U.S.C. § 112.

### Diluent

The compositions disclosed herein preferably include or are used with a diluent. The diluent may function as a carrier for the fertilizer compositions when provided in a liquid concentrate form. Additionally or alternatively, the diluent may be used to dilute a liquid concentrate or solid to form a “ready to use” use solution.

The diluent may comprise any suitable organic or inorganic liquid compound. Preferably, the diluent comprises water. Still more preferably, the diluent comprises de-chlorinated or distilled water. The diluent may be present in any suitable amount based on the desired dilution ratio, including between about 1% w/w/ to about 99% w/w.

### Additives

The compositions described herein may optionally further comprise one or more additives. Suitable additives include, without limitation, an anti-corrosion agent, anti-caking agent, stabilizer, anti-freeze, anti-foam agent, sticking agent, spreading agent, wetting agent, drift control agent, complexing agent, softening agent, a synthetic fertilizer (such as nitrogen or phosphorous), an immune system enhancer, an additional source of primary nutrients or secondary nutrients or micronutrients, or a combination thereof.

Examples of an additional source of primary nutrients or secondary nutrients or micronutrients include sources providing phosphorus or potassium as primary nutrients, and calcium, magnesium, or sulfur as secondary nutrients or micronutrients such as boron, copper, iron, manganese, molybdenum and zinc, or urea providing nitrogen.

Examples of suitable immune system enhancers (also referred to as plant immune system enhancers or boosters) include, without limitation, salicylic acid, chitosan, potassium (e.g., potassium sulfate), or a combination thereof.

In a preferred embodiment, the compositions further comprise a plant immune system enhancer comprising chitosan.

The one or more additives may be present in addition to the components of the composition described herein, either individually or in sum, in an amount of from about 0% w/w to about 50 w/w %, including between about 0.01% w/w to about 30% w/w, and between about 0.1% w/w to about 15% w/w, and between about 1% w/w and about 3% w/w, inclusive of all integers within these ranges.

### Methods of Making Fertilizer Compositions

The present disclosure also provides methods of making fertilizer compositions comprising stabilized microbial consortia. As described herein, consortia microorganisms have been successfully co-fermented and stabilized, offering direct growth and yield benefits to plants.

The methods disclosed herein involve a first step of generating a microbial consortium, for example the three Example consortia described herein. The method of generating a microbial consortium can comprise culturing one or more microorganisms; fermenting the one or more microorganisms to form a fermented mixture; and optionally separating the fermented mixture into solid and liquid fractions.

Culturing involves the intentional growth of one or more organisms or cells in the presence of assimilable sources of carbon, nitrogen, and relevant minerals and nutrients. In an example, such growth can take place in a solid or semi-solid nutritive medium, or in a liquid medium in which the nutrients are dissolved or suspended. In a further example, the culturing may take place on a surface or by submerged culture.

Fermenting involves a process that results in the breakdown of complex organic compounds into simpler compounds by microorganisms (such as bacteria and/or fungi). The fermentation process may occur under aerobic conditions, anaerobic conditions, or both (for example, in a large volume where some portions are aerobic and other portions are anaerobic). In some examples, the one or more microorganisms are incubated at a temperature of about 20-40° C. (for example, about 30°−35° C., such as about 30° C., about 31° C., about 32° C., about 33° C., about 34° C., about 35° C., about 36° C., about 37° C., about 38° C., about 39° C., or about 40° C.) for about 1 day to 30 days (such as about 2-28 days, about 4-24 days, about 16-30 days, about 10-20 days, or about 12-24 days). In some examples, the microorganisms are agitated periodically (for example, non-continuous agitation). In other examples, the microorganisms are continuously agitated. The pH of the fermentation mixture may be monitored periodically.

The fermented mixture is then optionally fractioned into solids and liquids. This separation can occur by any suitable method, for example decanting, filtration, and/or centrifugation. In an example, the fermented mixture is passed from the tank to settling equipment. The liquid is subsequently decanted and centrifuged. In one non-limiting example, the fermented mixture is centrifuged at 1250 rpm (930×g) for 15 minutes at about 5° C. to obtain liquid and lipid (e.g., pigment) fractions. The liquid (or aqueous) fraction obtained from the biodegradation process can be stored at ambient temperature.

More particularly, the microbial consortia are cultivated using sterilized water and blackstrap molasses as growth media. The consortia are first cultivated in smaller sizes (e.g., 10-20 oz). The small batches were cultivated at between about 65° F. to about 75° F., preferably about 70° F., with no direct sunlight, and preferably in darkness, for a period of between about 1 week to 4 weeks. The small batches are then added to larger, industrial scale consortia to ensure that the microbes in the consortia remain consistent across multiple batches. These larger batches are then brewed/fermented.

To address the problem of malodor and generation of undesirable gases, it was found that reducing the brewing/fermenting time from >48 hours-72 hours, to between 24-48 hours together with several formulation modifications surprisingly stabilized the microbial consortia such that the fertilizer compositions did not produce malodors or gasses, or result in a substantial reduction in cell viability. The formulation modifications included (1) adding a probiotic comprising 0.1 to 0.3 kg of Lactobacillus to the example formulations described herein; and (2) removing milk (pasteurized or raw), fermented starch water, and a calcium extract comprised of eggshells with vinegar, all of which were present in the first generation product.

Increasing brewing time or removing the probiotic is undesirable, as the shorter brewing time and presence of the probiotic beneficially and unexpectedly stabilizes the microbial consortia such that the consortia does not interact with the other components of the fertilizer composition or environmental elements and does not lose cell viability, explode, or produce malodors during storage and transportation.

Also disclosed herein are methods of making a fertilizer comprising a microbial consortium. Such a method involves combining one or more digestates with a seaweed, a cellulose source, one or more nutrient supplements, one or more humic substances, and a probiotic to form a base fertilizer composition; combining the base fertilizer composition with the microbial consortium to form a microbial fertilizer; and fermenting the microbial fertilizer. The method optionally further comprises a step of aerating the microbial fertilizer during the fermenting step. Such aeration can be done using any suitable device, such as an aeromixer. In an embodiment, the fermenting step lasts for between about 24 hours and about 48 hours, preferably between about 24 hours and about 36 hours.

### Methods of Treating Soil, Plants, Plant Tissue, Seedlings and Seeds

The present disclosure also provides methods of treating soil, plants, seedlings, and/or seeds and other plant tissues or parts. The fertilizer compositions described herein may be used to treat soil, plants, plant tissue, or plant parts (such as roots, stems, foliage, seeds, or seedlings). In some examples, treatment with the fertilizer compositions described herein provides improved plant growth, stress tolerance and/or crop yield.

In some embodiments the method of treating comprises contacting soil, a plant, a plant tissue, or a plant part with the compositions described herein to form a treated product. In an embodiment, the plant part comprises foliage, a stem, root, seedling, seed, or a combination thereof, and the treated product comprises soil, a plant, a plant tissue, or a plant part. The methods optionally further comprise a step of growing a treated product (e.g., plants, plant parts, or seeds and/or cultivating plants, plant parts or seeds) in the treated soil.

The contacting may last for any suitable time period, for example 1 minute, 5 minutes, 10 minutes, 30 minutes, 1 hour, 2 hours, 3 hours, 4 hours, 6 hours, 8 hours, 12 hours, 16 hours, 18 hours, or 24 hours. The contacting step may be optionally repeated one or more times. The time in between each contacting step may vary depending on the particular plant or growth environment. For example, the time between contacting steps may be at least 36 hours, at least 48 hours, at least 72 hours, at least 96 hours, at least one week, at least two weeks, at least four weeks, at least eight weeks, or at least 12 weeks.

In some examples, the amount of the fertilizer compositions to be applied will vary depending on the type of plant and growth environment. For example, for bulk applications (for example, per acre or hectare) the concentrate fertilizer compositions may be diluted in water to an amount sufficient to spray or irrigate the bulk area to be treated. In other examples, the fertilizer composition can be mixed with diluted herbicides, insecticides, or pesticides, and subsequently applied. If the composition to be applied is a solid, the solid can be applied directly to the soil, plants, or plant parts or can be suspended or dissolved in water (or other liquid) prior to use. In some embodiments, the fertilizer compositions can be applied to a soil, plant, plant tissue, or plant part by way of a spray, a drip, or by dipping all or part of the plant/plant part (e.g., seed) in the fertilizer composition. The compositions can be applied using a nozzle, hose, spray bottle, drip irrigation, or the like.

The fertilizer compositions can be delivered at different developmental stages of the plant, depending on the plant and agricultural practices. For example, the compositions may be applied onto seeds before planting, at the time of seed planting, and/or also applied to the soil near the roots at multiple times during the plant growth. In still further examples, the compositions are delivered through drip irrigation at low concentration at the seedlings stage or as transplants are being established, delivered in flood irrigation, or dispensed as a diluted mixture with nutrients in overhead or drip irrigation in greenhouses to seedlings or established plants, or are applied manually.

In some examples, treatment of soil, seeds, plants, or plant parts with a composition increases plant growth (such as overall plant size, amount of foliage, root number, root diameter, root length, production of tillers, fruit production, pollen production, or seed production) by at least about 5% (for example, at least about 10%, at least about 30%, at least about 50%, at least about 75%, or about 100%), inclusive of all integers up to 100%. Other measures of crop performance include quality of the plant, yield, pollination and fruit set, bloom, flower number, flower lifespan, bloom quality, rooting and root mass, crop resistance to lodging, pest resistance, abiotic stress tolerance to heat, drought, cold and recovery after stress, adaptability to poor soils, level of photosynthesis and greening, and plant health.

The disclosed methods and compositions can be used in connection with any plant, including forage crops, fruits, vegetables, grains, house plants, flowering plants, ornamental plants, and the like.

### EXAMPLES

### Example 1. Treatment with Fertilizer Compositions to Improve Plant Health

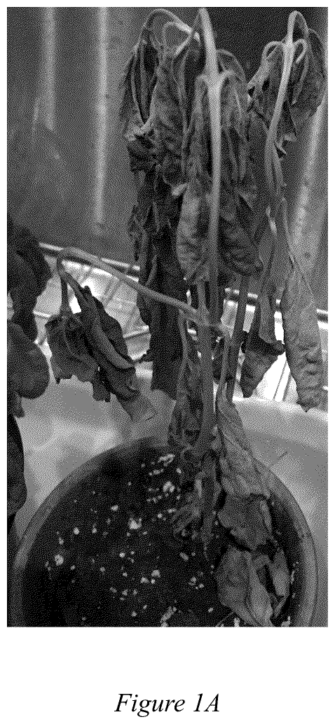

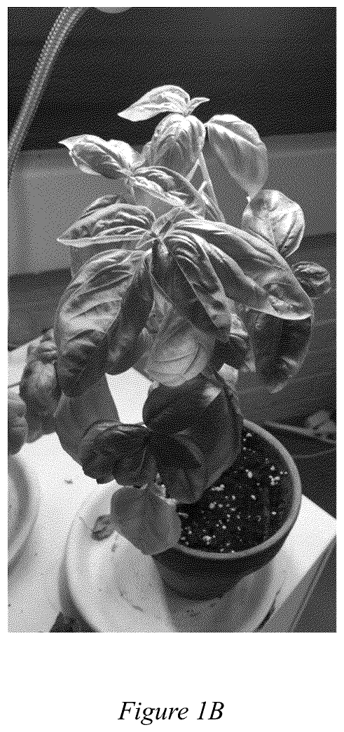

Several household plants at various stages of development were treated with Example 2 of Table 1B. The plants were treated with Example 2 of Table 1B diluted to a concentration of approximately 2 ounces per gallon once every 10 days, except for succulents, which were treated with a concentration of 2 ounces per gallon of water once a month. After 2-3 weeks of treatment, the health and appearance of the plants was compared before treatment with Example 2 of Table 1B and after treatment with Example 2 of Table 1B. The results are shown in FIGS. 1A-4.

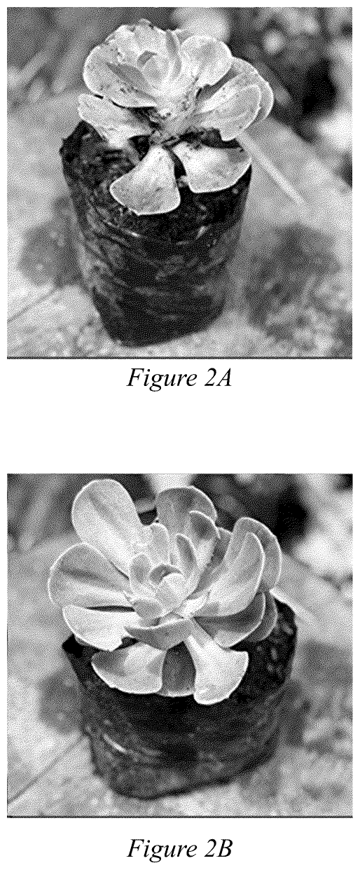

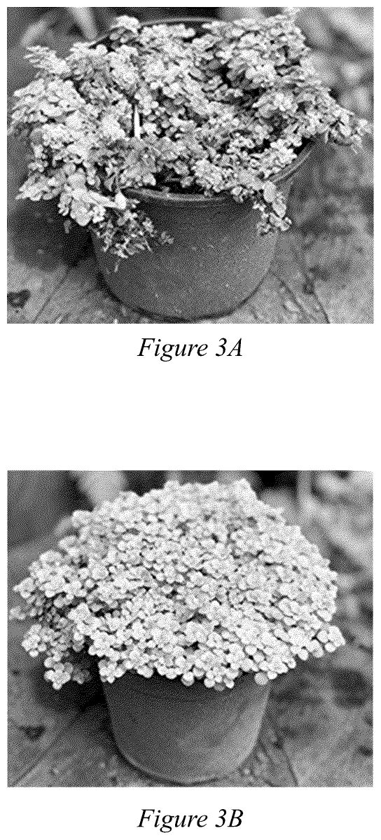

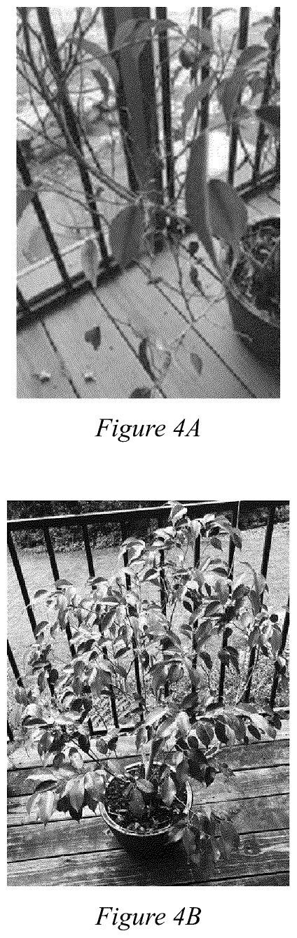

As shown in FIGS. 1A and 1B, the basil plant of FIG. 1A demonstrated significant wilting, yellowing of the leaves, and leaf loss. After treatment with Example 2 of Table 1B, leaf color and wilting were substantially improved. As shown in FIGS. 2A and 2B, the first succulent had poor leaf development, with some leaves having brown spots. After treatment with Example 2 of Table 1B, the succulent demonstrated significant improvement in leaf quality, namely leaf size, color, and the elimination of brown spots. As shown in FIGS. 3A-3B, a second succulent showing wilting and browning was treated with Example 2 of Table 1B. After treatment, the second succulent demonstrated a substantial reduction in browning and a significant increase in leaf growth. Finally, as shown in FIGS. 4A and 4B, the Ficus plant treated with Example 2 of Table 1B demonstrated a significant increase in leaf growth and health, as well as a reduction in yellowing.

### Example 2. Effective Stabilization of Microbial Consortia

A key challenge in developing bioactive fertilizer compositions is the loss of microbial viability, particularly during transport and storage. In addition to reduced efficacy as a fertilizer, compositions with loss of microbial viability also have undesirable properties, such as foul odor and the generation of gas byproducts, which can lead to container distortion or explosion.

The fertilizer compositions described herein beneficially stabilize the microbial consortia, as evidenced by good cell viability and a lack of malodor and gases. For any composition comprising live microbes, it is challenging to stabilize the compositions such that the microbes remain viable and do not negatively interact with the components of the fertilizer composition or other elements of the environment. For example, earlier iterations of the fertilizer compositions undesirably produced malodors and excessive gasses to the extent that containers comprising the fertilizer compositions would explode during storage or transport.

More particularly, the microbial consortia were cultivated using sterilized water and blackstrap molasses as growth media. The consortia were first cultivated in smaller sizes (e.g., 10-20 oz). The small batches were cultivated at approximately 70° F. with no direct sunlight for a period of between 1 week to 4 weeks. The small batches were then added to larger, industrial scale consortia to ensure that the microbes in the consortia remained consistent across multiple batches. These larger batches were then brewed/fermented.

To address the problem of malodor and generation of undesirable gases, it was found that reducing the brewing/fermenting time from >48 hours-72 hours, to between 24-48 hours together with several formulation modifications surprisingly stabilized the microbial consortia such that the fertilizer compositions did not produce malodors or gasses, or result in a substantial reduction in cell viability. The formulation modifications included (1) adding a probiotic comprising 0.1 to 0.3 kg of Lactobacillus to the example formulations described herein; and (2) removing milk (pasteurized or raw), fermented starch water, and a calcium extract comprised of eggshells with vinegar, all of which were present in the first generation product.

TABLE 8
Stabilization of Microbial Consortia and Fertilizer Compositions

| | | | Demonstrated |
|---|---|---|---|
| | Container | | Loss of Cell |
| Product | Explosions? | Malodors? | Viability? |
| First Generation Product | Yes | Yes | Yes |
| (brewed >48 hrs-72 hrs) | | | |
| Example 2.2 of Table 1B | No | No | No |
| (24 hr brew) | | | |
| Example 2.2 of Table 1B | No | No | No |
| (48 hr brew) | | | |
| Example 2.2 of Table 2B | No | No | No |
| (24-48 hr brew) | | | |

### Example 3. Corn Case Study

The formulations described herein were evaluated for their ability to improve corn growth, health, and pest resistance. A fertilizer composition was prepared according to the formulations described herein. In particular, test formulation “50/50” was prepared comprising about 50% w/w (e.g., 49.5%) of Example 2 (as shown in Table 1B) and about 50% w/w (e.g., 49.5%) of Example 4 (as shown in Table 2B) plus 1% chitosan, and applied to a corn field together with 30 lbs synthetic nitrogen (5 gallons 50/50 in furrow). 150 lbs of synthetic nitrogen was applied to a second comparison corn field.

The 50/50 formulation applied in conjunction with the nitrogen produced a comparable/slightly improved yield to a field with 80% more synthetic nitrogen (on average 130 bushes per acre) resulting in a reduction of fertilizer input costs of 50%. In particular, yield maps showed areas in the field treated with 50/50 as having 180-220 bushels per acre in comparison to the highest recorded yield historically of 150 bushels.

Additional benefits of the 50/50 formulation were observed. Specifically, the corn in the field treated with 50/50 demonstrated 50% less disease spots, and 75% fewer Japanese beetles. More broadly, the 50/50 formulation decreased input costs, as the formulation locked in nutrients at the plant roots, boosting the effectiveness the synthetic nitrogen, and reducing the volume of nitrogen fertilizer needed to achieve the same yield. Additionally, the viable microbes in the 50/50 formulation continue to nitrogen fix and phosphorus solubilize throughout the growing season.

### Example 4. Soybean Case Study

The formulations described herein were evaluated for their ability to improve soybean growth and health. A fertilizer composition was prepared according to the formulations described herein. In particular, test formulation “75/25” was prepared comprising about 75% w/w (e.g., 74.5%) of Example 4 (as shown in Table 2B), and about 25% w/w (e.g., 24.5%) of Example 2 (as shown in Table 1B), plus 1% chitosan, and applied to a soybean field at a concentration of approximately 2.5 gallons per acre of soybeans. Synthetic nitrogen was applied to a second comparison soybean field.

Overall, the yield was 82 bushels per acre for the field treated with 75/25. These yields were within 2-3 bushes per acre of the synthetic nitrogen field. The field treated with 75/25 therefore provided a 60% cost savings on fertilizer compared to the synthetic nitrogen field.

Additional benefits of the 75/25 formulation were observed. Specifically, the soybeans in the field treated with 75/25 formulation decreased input costs, as the formulation locked in nutrients at the plant roots, boosting the effectiveness the synthetic nitrogen, and reducing the volume of nitrogen fertilizer needed to achieve the same yield. Additionally, the viable microbes in the 75/25 formulation continue to nitrogen fix and phosphorus solubilize throughout the growing season.

The embodiments being thus described, it will be obvious that the same may be varied in many ways. Such variations are not to be regarded as a departure from the spirit and scope of the disclosure and all such modifications are intended to be included within the scope of the following claims.

What is claimed is:

1. A microbial fertilizer composition comprising:
one or more digestates;
a seaweed;
one or more cellulose sources;
one or more nutrient supplements;
one or more humic substances;
a probiotic; and
a microbial consortium comprising one or more microorganisms from the phyla proteobacteria, firmicutes, bacteroidota, actinobacteria, campylobacterota, verrucomicrobiota, desulfobacterota, ascomycota, mortierellomycota, basidiomycota, mucoromycota, olpidiomycota, or a combination thereof.

2. The microbial fertilizer composition of claim 1, wherein the one or more digestates comprise vermicompost, seabird guano, or a combination thereof.

3. The microbial fertilizer composition of claim 1, wherein the seaweed comprises green algae, brown algae, red algae, or a combination thereof.

4. The microbial fertilizer composition of claim 1, wherein the one or more cellulose sources comprise alfalfa, comfrey, nettles, yarrow, yucca, agave, or a combination thereof.

5. The microbial fertilizer composition of claim 1, wherein the one or more nutrient supplements comprise bone meal, fish meal, magnesium sulfate, calcium sulfate, silicon dioxide, potassium silicate, calcium silicate silicic acid, or a combination thereof.

6. The microbial fertilizer composition of claim 1, wherein the one or more humic substances comprise humins, humic acids, fulvic acids, or a combination thereof.

7. The microbial fertilizer composition of claim of claim 1, wherein the probiotic comprises lactic acid bacteria, yeast, phototrophic bacteria, or a combination thereof.

8. The microbial fertilizer composition of claim 1, wherein the microbial consortium comprises NRRL No. XXXXX, NRRL No. YYYYY, NRRL No. ZZZZZ, Oryzomicrobium terrae, Cloacibacterium sp., Acinetobacter brisouii, Dysgonomonas mossii, Acinetobacter calcoaceticus, Acetobacteroides hydrogenigenes, Prevotella sp., Sedimentibacter sp., Azotobacter chroococcum, Prevotella paludivivens, Comamonas testosteroni, Lentilactobacillus sp., Acetobacter syzygii, Leuconostoc mesenteroides, Bacteroides sp., Arcobacter butzleri, Xanthomonas massiliensis, Microvirgula aerodenitrificans, Lactococcus lactis, Bifidobacterium mongoliense, Dechlorosoma suillum, Clostridium sp., Citrobacter freundii, Bacteroides luti, Bifidobacterium psychraerophilum, Pseudomonas sp., Lactobacillus paracasei, Salmonella enterica, Dysgonomonas sp., Desulfosporosinus sp., Acinetobacter junii, Comamonas sp., Azoarcus indigens, Chthoniobacter sp., Clostridium magnum, Lactobacillus harbinensis, Neobacillus drentensis, Clostridium methoxybenzovorans, Desulfovibrio sp., Colidextribacter sp., Comamonas aquatica, Lachnoclostridium sp., Clostridium saccharolyticum, Desulfosporosinus hippei, Sporomusa sp., Fonticella sp., Anaerovorax sp., Acinetobacter haemolyticus, Lactobacillus perolens, Desulfitobacterium sp., Mobilitalea sp., Desulfitobacterium dichloroeliminans, Achromobacter xylosoxidans, Clostridium sporosphaeroides, Acinetobacter sp., Rhodobacter sp., Lactobacillus bifermentans, Paenirhodobacter enshiensis, Hydrogenoanaerobacterium sp., Clostridium beijerinckii, Stenotrophomonas maltophilia, Elizabethkingia meningoseptica, Acinetobacter kookii, Stenotrophomonas acidaminiphila, Clostridium viride, Enterococcus casseliflavus, Sporomusa ovata, Desulfotomaculum defluvii, Pedobacter tournemirensis, Nitrosotenuis sp., Oscillibacter sp., Chryseobacterium taihuense, Ruminiclostridium sp., Ruminococcus sp., Anaerocolumna sp., Escherichia coli, Bacillus sp., Amnipila sp., Reyranella sp., Leuconostoc pseudomesenteroides, Comamonas terrigena, Citrobacter amalonaticus, Enterococcus italicus, Lactobacillus vaccinostercus, Butyricicoccus sp., Monoglobus sp., Kosakonia oryzae, Micropepsis sp., Clostridium tunisiense, Dechlorosoma sp., Ilyobacter delafieldii, Lacticaseibacillus sp., Udaeobacter sp., Delftia sp., Ruminiclostridium hungatei, Sporomusa silvacetica, Nitrosocosmicus oleophilus, Desulfotomaculum sp., Desulfosporomusa polytropa, Acidipropionibacterium acidipropionici, Caproiciproducens sp., Pandoraea pnomenusa, Actinomyces sp., Herbaspirillum huttiense, Phenylobacterium sp., Papillibacter sp., Saccharibacillus sp., Pseudomonas otitidis, Schaalia turicensis, Desulfosporosinus fructosivorans, Novosphingobium resinovorum, Lactobacillus delbrueckii, Pseudomonas putida, Anaerocolumna xylanovorans, Pseudomonas aeruginosa, Acinetobacter variabilis, Caulobacter sp., Clostridium subterminale, Acinetobacter ursingii, Oxobacter sp., Acetanaerobacterium sp., Clostridium intestinale, Lactobacillus pentosus, Sporomusa malonica, Rhodovastum sp., Clostridium homopropionicum, Paracoccus sp., Clostridium malenominatum, Anaerostignum sp., Aeromonas hydrophila, Desulfosporosinus meridiei, Acetobacter sp., Pseudarthrobacter oxydans, Azospirillum sp., Citrobacter sp., Pseudomonas stutzeri, Herbinix sp., Stenotrophomonas sp., Serratia marcescens, Acinetobacter radioresistens, Desulfosporosinus youngiae, Desulfofarcimen sp., Trabulsiella sp., Oxalophagus oxalicus, Clostridium tyrobutyricum, Enterococcus sp., Tabrizicola sp., Hydrogenophaga sp., Novosphingobium sp., Neorhizobium alkalisoli, Methylophilus sp., Desulfobulbus sp., Geminisphaera sp., Paludibacter sp., Solimonas flava, Flavobacterium sp., Pleomorphomonas sp., Sphingobium sp., Shewanella dokdonensis, Klebsiella pneumoniae, Rhizobium sp., Pseudacidovorax intermedius, Magnetospirillum sp., Siphonobacter aquaeclarae, Oleomonas sagaranensis, Riegeria sp., Quatrionicoccus sp., Paenirhodobacter sp., Ideonella dechloratans, Roseomonas sp., Xanthobacter polyaromaticivorans, Thiobacillus thioparus, Ancalomicrobium sp., Uliginosibacterium sp., Anaerosporobacter sp., Pseudomonas oleovorans, Devosia insulae, Agrobacterium tumefaciens, Erythrobacter mathurensis, Propionivibrio sp., Lacunisphaera sp., Chitinophaga pinensis, Xanthobacter autotrophicus, Mucilaginibacter litoreus, Dyadobacter fermentans, Brevundimonas aurantiaca, Bosea sp., Chitinophaga sancti, Xanthobacter flavus, Sphingobium yanoikuyae, Desulfovibrio vulgaris, Azoarcus olearius, Catellibacterium terrae, Rhodovarius lipocyclicus, Aquaspirillum polymorphum, Roseomonas rubra, Starkeya novella, Paracoccus yeei, Gracilibacter sp., Dechlorosoma suillum, Aeromonas sp., Acinetobacter johnsonii, Anaerotaenia torta, Parabacteroides chartae, Pedobacter rhizosphaerae, Anaerocolumna cellulosilytica, Rhizobium undicola, Shinella zoogloeoides, Bdellovibrio sp., Propionicimonas sp., Lachnotalea sp., Bosea thiooxidans, Variovorax ginsengisoli, Kaistia hirudinis, Zoogloea sp., Kaistia dalseonensis, Opitutus sp., Kaistia sp., Terrimicrobium sacchariphilum, Mycolicibacterium mucogenicum, Asticcacaulis sp., Rhizobium gallicum, Cellulosimicrobium sp., Prosthecobacter debontii, Mycobacterium gilvum, Sphingomonas wittichii, Cereibacter changlensis, Pseudomonas mendocina, Variovorax sp., Pseudaeromonas pectinilytica, Cupriavidus campinensis, Bacillus circulans, Fibrisoma sp., Moraxella osloensis, Ensifer adhaerens, Rhizobium rhizoryzae, Novosphingobium aromaticivorans, Shinella fusca, Roseomonas eburnea, Roseomonas cervicalis, Acinetobacter soli, Rhodoplanes roseus, Paenibacillus ihuae, Caulobacter mirabilis, Enterobacter sp., Flavobacterium akiainvivens, Pseudomonas alcaligenes, Paenibacillus graminis, Marinomonas sp., Xinfangfangia soli, Imtechium assamiensis, Raoultella planticola, Roseomonas aestuarii, Legionella lytica, Novosphingobium sediminicola, Yersinia pestis, Raoultella ornithinolytica, Enterobacter hormaechei, Mycoplana sp., Pseudomonas nitroreducens, Rhizobium petrolearium, Shinella kummerowiae, Propionispora vibrioides, Enterobacter cancerogenus, Ochrobactrum intermedium, Klebsiella sp., Acinetobacter tandoii, Cloacibacterium haliotis, Lactiplantibacillus sp., Yersinia bercovieri, Pelosinus sp., Acinetobacter lwoffii, Anaerospora sp., Lactobacillus coryniformis, Pseudomonas psychrotolerans, Agrobacterium albertimagni, Acinetobacter venetianus, Cyberlindnera jadinii, Penicillium herquei, Williopsis sp., Mortierella hyalina, Pichia kudriavzevii, Mucor circinelloides, Candida tropicalis, Preussia flanaganii, Mortierella sp., Penicillium sp., Penicillium melinii, Mortierella gamsii, Starmera stellimalicola, Purpureocillium lavendulum, Mucor irregularis, Monocillium indicum, Penicillium griseofulvum, Solicoccozyma aeria, Mortierella alpina, Monocillium mucidum, Mortierella ambigua, Tetracladium sp., Tausonia pullulans, Trichoderma piluliferum, Stropharia coronilla, Gymnostellatospora japonica, Aspergillus caninus, Meyerozyma caribbica, Penicillium bilaiae, Trichoderma harzianum, Pseudogymnoascus roseus, Penicillium oxalicum, Talaromyces marne ei, Phaeosphaeriopsis sp., Penicillium brevicompactum, Alternaria longissima, Candida parapsilosis, Pseudostrickeria sp., Allophoma tropica, Leptosphaeria maculans, Trichoderma pubescens, Cortinarius helvolus, Mortierella sarnyensis, Pyrenochaetopsis leptospora, Exophiala sp., Pseudothielavia terricola, Cladosporium herbarum, Trichoderma asperellum, Aspergillus fumigatus, Alternaria eichhorniae, Talaromyces piceae, Penicillium levitum, Talaromyces sp., Chrysosporium lobatum, Didymella exigua, Aspergillus chlamydosporus, Penicillium roseopurpureum, Subramaniula asteroides, Aspergillus pseudodeflectus, Penicillium citrinum, Chrysosporium pseudomerdarium, Mortierella rishikesha, Neobulgaria sp., Curvularia verruculosa, Clavaria sp., Plectosphaerella cucumerina, Mortierella minutissima, Penicillium parviverrucosum, Panaeolus fimicola, Psathyrella ichnusae, Arachnomyces gracilis, Solicoccozyma terrea, Mortierella antarctica, Coniothyrium cerealis, Neodactylaria sp., Penicillium pimiteouiense, Penicillium simplicissimum, Humicola olivacea, Arachnomyces kanei, Mortierella lignicola, Thelebolus globosus, Bipolaris microlaenae, Penicillium erubescens, Polyschema sclerotigenum, Phaeosphaeria sp., Sarocladium strictum, Penicillium restrictum, Fusarium proliferatum, Penicillium argentinense, Alternaria metachromatica, Coniothyrium sp., Talaromyces purpureogenus, Aspergillus niger, Lachnum sp., Gymnopus sp., Penicillium rubens, Alternaria photistica, Sporormiella megalospora, Didymella pinodes, Phallus rugulosus, Coniochaeta canina, Cutaneotrichosporon jirovecii, Aspergillus ochraceus, Penicillium catenatum, Microscypha sp., Sebacina sp., Talaromyces stollii, Oidiodendron cereale, Hannaella oryzae, Furcasterigmium furcatum, Fusarium solani, Clonostachys rosea, Aspergillus wentii, Kernia pachypleura, Olpidium brassicae, Penicillium steckii, Byssochlamys lagunculariae, Acrocalymma vagum, Cephalotrichum microsporum, Stemphylium vesicarium, Leohumicola verrucosa, Aspergillus subversicolor, Wallemia sebi, Trichosporon asahii, Rhizoctonia solani, Actinomucor elegans, Scedosporium dehoogii, Scedosporium boydii, Gibellulopsis serrae, Vishniacozyma victoriae, Cunninghamella echinulata, Chaetosphaeronema sp., Chrysosporium sp., Filobasidium magnum, Epicoccum thailandicum, Aspergillus sp., Oidiodendron echinulatum, Penicillium polonicum, Oedocephalum nayoroense, Fusarium oxysporum, Hirsutella minnesotensis, Aspergillus flavus, Penicillium atrovenetum, Chrysosporium synchronum, Hymenoscyphus menthae, Talaromyces radicus, Mortierella elongata, Microascus trigonosporus, Humicola fuscoatra, Vishniacozyma tephrensis, Aspergillus terreus, Neosetophoma samarorum, Aureobasidium pullulans, Wallemia canadensis, Circinella muscae, Botrytis caroliniana, Aspergillus penicillioides, Aspergillus rugulosus, Preussia sp., Malbranchea flocciformis, Malbranchea cinnamomea, Microdochium sp., Fusarium pseudensiforme, Chaetomium acropullum, Vishniacozyma globispora, or a combination thereof.

9. The microbial fertilizer composition of claim 1, wherein the composition comprises from about 5 wt. % to about 75 wt. % of the one or more digestates, from about 5 wt. % to about 45 wt. % of the seaweed, from about 0.001 wt. % to about 30 wt. % of the one or more cellulose sources, from about 0.01 wt. % to about 90 wt. % of the nutrient supplements, from about 0.01 wt. % to about 0 wt. % of the of the humic substances, from about 0.01 wt. % to about 5 wt. % of the probiotic, and from about 0.01 wt. % to about 5 wt. % of the microbial consortium.

10. The microbial fertilizer composition of claim 1, wherein the composition further comprises an additive comprising an anti-corrosion agent, anti-caking agent, stabilizer, anti-freeze, anti-foam agent, sticking agent, spreading agent, wetting agent, drift control agent, complexing agent, softening agent, an immune system enhancer, an additional source of primary nutrients or secondary nutrients or micronutrients, or a combination thereof.

11. A method of generating a microbial consortium comprising:
culturing one or more microorganisms comprising proteobacteria, firmicutes, bacteroidota, actinobacteria, campylobacterota, verrucomicrobiota, desulfobacterota, ascomycota, mortierellomycota, basidiomycota, mucoromycota, olpidiomycota, or a combination thereof to form a microbial culture;
adding a probiotic to the microbial culture;
fermenting the microbial culture for between about 24 hours to about 48 hours to form a microbial consortium; and
optionally separating the microbial consortium into solid and liquid fractions.

12. The method of claim 11, wherein the microbial consortium comprises NRRL No.
XXXXX, NRRL No. YYYYY, NRRL No. ZZZZZ, Oryzomicrobium terrae, Cloacibacterium sp., Acinetobacter brisouii, Dysgonomonas mossii, Acinetobacter calcoaceticus, Acetobacteroides hydrogenigenes, Prevotella sp., Sedimentibacter sp., Azotobacter chroococcum, Prevotella paludivivens, Comamonas testosteroni, Lentilactobacillus sp., Acetobacter syzygii, Leuconostoc mesenteroides, Bacteroides sp., Arcobacter butzleri, Xanthomonas massiliensis, Microvirgula aerodenitrificans, Lactococcus lactis, Bifidobacterium mongoliense, Dechlorosoma suillum, Clostridium sp., Citrobacter freundii, Bacteroides luti, Bifidobacterium psychraerophilum, Pseudomonas sp., Lactobacillus paracasei, Salmonella enterica, Dysgonomonas sp., Desulfosporosinus sp., Acinetobacter junii, Comamonas sp., Azoarcus indigens, Chthoniobacter sp., Clostridium magnum, Lactobacillus harbinensis, Neobacillus drentensis, Clostridium methoxybenzovorans, Desulfovibrio sp., Colidextribacter sp., Comamonas aquatica, Lachnoclostridium sp., Clostridium saccharolyticum, Desulfosporosinus hippei, Sporomusa sp., Fonticella sp., Anaerovorax sp., Acinetobacter haemolyticus, Lactobacillus perolens, Desulfitobacterium sp., Mobilitalea sp., Desulfitobacterium dichloroeliminans, Achromobacter xylosoxidans, Clostridium sporosphaeroides, Acinetobacter sp., Rhodobacter sp., Lactobacillus bifermentans, Paenirhodobacter enshiensis, Hydrogenoanaerobacterium sp., Clostridium beijerinckii, Stenotrophomonas maltophilia, Elizabethkingia meningoseptica, Acinetobacter kookii, Stenotrophomonas acidaminiphila, Clostridium viride, Enterococcus casseliflavus, Sporomusa ovata, Desulfotomaculum defluvii, Pedobacter tournemirensis, Nitrosotenuis sp., Oscillibacter sp., Chryseobacterium taihuense, Ruminiclostridium sp., Ruminococcus sp., Anaerocolumna sp., Escherichia coli, Bacillus sp., Amnipila sp., Reyranella sp., Leuconostoc pseudomesenteroides, Comamonas terrigena, Citrobacter amalonaticus, Enterococcus italicus, Lactobacillus vaccinostercus, Butyricicoccus sp., Monoglobus sp., Kosakonia oryzae, Micropepsis sp., Clostridium tunisiense, Dechlorosoma sp., Ilyobacter delafieldii, Lacticaseibacillus sp., Udaeobacter sp., Delftia sp., Ruminiclostridium hungatei, Sporomusa silvacetica, Nitrosocosmicus oleophilus, Desulfotomaculum sp., Desulfosporomusa polytropa, Acidipropionibacterium acidipropionici, Caproiciproducens sp., Pandoraea pnomenusa, Actinomyces sp., Herbaspirillum huttiense, Phenylobacterium sp., Papillibacter sp., Saccharibacillus sp., Pseudomonas otitidis, Schaalia turicensis, Desulfosporosinus fructosivorans, Novosphingobium resinovorum, Lactobacillus delbrueckii, Pseudomonas putida, Anaerocolumna xylanovorans, Pseudomonas aeruginosa, Acinetobacter variabilis, Caulobacter sp., Clostridium subterminale, Acinetobacter ursingii, Oxobacter sp., Acetanaerobacterium sp., Clostridium intestinale, Lactobacillus pentosus, Sporomusa malonica, Rhodovastum sp., Clostridium homopropionicum, Paracoccus sp., Clostridium malenominatum, Anaerostignum sp., Aeromonas hydrophila, Desulfosporosinus meridiei, Acetobacter sp., Pseudarthrobacter oxydans, Azospirillum sp., Citrobacter sp., Pseudomonas stutzeri, Herbinix sp., Stenotrophomonas sp., Serratia marcescens, Acinetobacter radioresistens, Desulfosporosinus youngiae, Desulfofarcimen sp., Trabulsiella sp., Oxalophagus oxalicus, Clostridium tyrobutyricum, Enterococcus sp., Tabrizicola sp., Hydrogenophaga sp., Novosphingobium sp., Neorhizobium alkalisoli, Methylophilus sp., Desulfobulbus sp., Geminisphaera sp., Paludibacter sp., Solimonas flava, Flavobacterium sp., Pleomorphomonas sp., Sphingobium sp., Shewanella dokdonensis, Klebsiella pneumoniae, Rhizobium sp., Pseudacidovorax intermedius, Magnetospirillum sp., Siphonobacter aquaeclarae, Oleomonas sagaranensis, Riegeria sp., Quatrionicoccus sp., Paenirhodobacter sp., Ideonella dechloratans, Roseomonas sp., Xanthobacter polyaromaticivorans, Thiobacillus thioparus, Ancalomicrobium sp., Uliginosibacterium sp., Anaerosporobacter sp., Pseudomonas oleovorans, Devosia insulae, Agrobacterium tumefaciens, Erythrobacter mathurensis, Propionivibrio sp., Lacunisphaera sp., Chitinophaga pinensis, Xanthobacter autotrophicus, Mucilaginibacter litoreus, Dyadobacter fermentans, Brevundimonas aurantiaca, Bosea sp., Chitinophaga sancti, Xanthobacter flavus, Sphingobium yanoikuyae, Desulfovibrio vulgaris, Azoarcus olearius, Catellibacterium terrae, Rhodovarius lipocyclicus, Aquaspirillum polymorphum, Roseomonas rubra, Starkeya novella, Paracoccus yeei, Gracilibacter sp., Dechlorosoma suillum, Aeromonas sp., Acinetobacter johnsonii, Anaerotaenia torta, Parabacteroides chartae, Pedobacter rhizosphaerae, Anaerocolumna cellulosilytica, Rhizobium undicola, Shinella zoogloeoides, Bdellovibrio sp., Propionicimonas sp., Lachnotalea sp., Bosea thiooxidans, Variovorax ginsengisoli, Kaistia hirudinis, Zoogloea sp., Kaistia dalseonensis, Opitutus sp., Kaistia sp., Terrimicrobium sacchariphilum, Mycolicibacterium mucogenicum, Asticcacaulis sp., Rhizobium gallicum, Cellulosimicrobium sp., Prosthecobacter debontii, Mycobacterium gilvum, Sphingomonas wittichii, Cereibacter changlensis, Pseudomonas mendocina, Variovorax sp., Pseudaeromonas pectinilytica, Cupriavidus campinensis, Bacillus circulans, Fibrisoma sp., Moraxella osloensis, Ensifer adhaerens, Rhizobium rhizoryzae, Novosphingobium aromaticivorans, Shinella fusca, Roseomonas eburnea, Roseomonas cervicalis, Acinetobacter soli, Rhodoplanes roseus, Paenibacillus ihuae, Caulobacter mirabilis, Enterobacter sp., Flavobacterium akiainvivens, Pseudomonas alcaligenes, Paenibacillus graminis, Marinomonas sp., Xinfangfangia soli, Imtechium assamiensis, Raoultella planticola, Roseomonas aestuarii, Legionella lytica, Novosphingobium sediminicola, Yersinia pestis, Raoultella ornithinolytica, Enterobacter hormaechei, Mycoplana sp., Pseudomonas nitroreducens, Rhizobium petrolearium, Shinella kummerowiae, Propionispora vibrioides, Enterobacter cancerogenus, Ochrobactrum intermedium, Klebsiella sp., Acinetobacter tandoii, Cloacibacterium haliotis, Lactiplantibacillus sp., Yersinia bercovieri, Pelosinus sp., Acinetobacter lwoffii, Anaerospora sp., Lactobacillus coryniformis, Pseudomonas psychrotolerans, Agrobacterium albertimagni, Acinetobacter venetianus, Cyberlindnera jadinii, Penicillium herquei, Williopsis sp., Mortierella hyalina, Pichia kudriavzevii, Mucor circinelloides, Candida tropicalis, Preussia flanaganii, Mortierella sp., Penicillium sp., Penicillium melinii, Mortierella gamsii, Starmera stellimalicola, Purpureocillium lavendulum, Mucor irregularis, Monocillium indicum, Penicillium griseofulvum, Solicoccozyma aeria, Mortierella alpina, Monocillium mucidum, Mortierella ambigua, Tetracladium sp., Tausonia pullulans, Trichoderma piluliferum, Stropharia coronilla, Gymnostellatospora japonica, Aspergillus caninus, Meyerozyma caribbica, Penicillium bilaiae, Trichoderma harzianum, Pseudogymnoascus roseus, Penicillium oxalicum, Talaromyces marne ei, Phaeosphaeriopsis sp., Penicillium brevicompactum, Alternaria longissima, Candida parapsilosis, Pseudostrickeria sp., Allophoma tropica, Leptosphaeria maculans, Trichoderma pubescens, Cortinarius helvolus, Mortierella sarnyensis, Pyrenochaetopsis leptospora, Exophiala sp., Pseudothielavia terricola, Cladosporium herbarum, Trichoderma asperellum, Aspergillus fumigatus, Alternaria eichhorniae, Talaromyces piceae, Penicillium levitum, Talaromyces sp., Chrysosporium lobatum, Didymella exigua, Aspergillus chlamydosporus, Penicillium roseopurpureum, Subramaniula asteroides, Aspergillus pseudodeflectus, Penicillium citrinum, Chrysosporium pseudomerdarium, Mortierella rishikesha, Neobulgaria sp., Curvularia verruculosa, Clavaria sp., Plectosphaerella cucumerina, Mortierella minutissima, Penicillium parviverrucosum, Panaeolus fimicola, Psathyrella ichnusae, Arachnomyces gracilis, Solicoccozyma terrea, Mortierella antarctica, Coniothyrium cerealis, Neodactylaria sp., Penicillium pimiteouiense, Penicillium simplicissimum, Humicola olivacea, Arachnomyces kanei, Mortierella lignicola, Thelebolus globosus, Bipolaris microlaenae, Penicillium erubescens, Polyschema sclerotigenum, Phaeosphaeria sp., Sarocladium strictum, Penicillium restrictum, Fusarium proliferatum, Penicillium argentinense, Alternaria metachromatica, Coniothyrium sp., Talaromyces purpureogenus, Aspergillus niger, Lachnum sp., Gymnopus sp., Penicillium rubens, Alternaria photistica, Sporormiella megalospora, Didymella pinodes, Phallus rugulosus, Coniochaeta canina, Cutaneotrichosporon jirovecii, Aspergillus ochraceus, Penicillium catenatum, Microscypha sp., Sebacina sp., Talaromyces stollii, Oidiodendron cereale, Hannaella oryzae, Furcasterigmium furcatum, Fusarium solani, Clonostachys rosea, Aspergillus wentii, Kernia pachypleura, Olpidium brassicae, Penicillium steckii, Byssochlamys lagunculariae, Acrocalymma vagum, Cephalotrichum microsporum, Stemphylium vesicarium, Leohumicola verrucosa, Aspergillus subversicolor, Wallemia sebi, Trichosporon asahii, Rhizoctonia solani, Actinomucor elegans, Scedosporium dehoogii, Scedosporium boydii, Gibellulopsis serrae, Vishniacozyma victoriae, Cunninghamella echinulata, Chaetosphaeronema sp., Chrysosporium sp., Filobasidium magnum, Epicoccum thailandicum, Aspergillus sp., Oidiodendron echinulatum, Penicillium polonicum, Oedocephalum nayoroense, Fusarium oxysporum, Hirsutella minnesotensis, Aspergillus flavus, Penicillium atrovenetum, Chrysosporium synchronum, Hymenoscyphus menthae, Talaromyces radicus, Mortierella elongata, Microascus trigonosporus, Humicola fuscoatra, Vishniacozyma tephrensis, Aspergillus terreus, Neosetophoma samarorum, Aureobasidium pullulans, Wallemia canadensis, Circinella muscae, Botrytis caroliniana, Aspergillus penicillioides, Aspergillus rugulosus, Preussia sp., Malbranchea flocciformis, Malbranchea cinnamomea, Microdochium sp., Fusarium pseudensiforme, Chaetomium acropullum, Vishniacozyma globispora, or a combination thereof.

13. A method of making a microbial fertilizer composition comprising:
combining one or more digestates with a seaweed, a cellulose source, one or more nutrient supplements, one or more humic substances, and a probiotic to form a base fertilizer composition;
combining the base fertilizer composition with the microbial consortium of claim 1 to form a microbial fertilizer; and
fermenting the microbial fertilizer.

14. The method of claim 13, further comprising a step of aerating the microbial fertilizer during the fermenting step.

15. The method of claim 13, wherein the fermenting step lasts for between about 24 hours and about 48 hours.

16. A method of treating a soil, plant, plant tissue, or plant part comprising:
contacting a soil, plant, plant tissue, plant part, or a combination thereof with a microbial fertilizer composition comprising one or more digestates, a seaweed, one or more cellulose sources, one or more nutrient supplements, one or more humic substances, a probiotic, and a microbial consortium comprising one or more microorganisms from the phyla proteobacteria, firmicutes, bacteroidota, actinobacteria, campylobacterota, verrucomicrobiota, desulfobacterota, ascomycota, mortierellomycota, basidiomycota, mucoromycota, olpidiomycota, or a combination thereof to form a treated product; and
optionally, repeating the contacting.

17. The method of claim 16, wherein the microbial consortium comprises NRRL No. XXXXX, NRRL No. YYYYY, NRRL No. ZZZZZ, Oryzomicrobium terrae, Cloacibacterium sp., Acinetobacter brisouii, Dysgonomonas mossii, Acinetobacter calcoaceticus, Acetobacteroides hydrogenigenes, Prevotella sp., Sedimentibacter sp., Azotobacter chroococcum, Prevotella paludivivens, Comamonas testosteroni, Lentilactobacillus sp., Acetobacter syzygii, Leuconostoc mesenteroides, Bacteroides sp., Arcobacter butzleri, Xanthomonas massiliensis, Microvirgula aerodenitrificans, Lactococcus lactis, Bifidobacterium mongoliense, Dechlorosoma suillum, Clostridium sp., Citrobacter freundii, Bacteroides luti, Bifidobacterium psychraerophilum, Pseudomonas sp., Lactobacillus paracasei, Salmonella enterica, Dysgonomonas sp., Desulfosporosinus sp., Acinetobacter junii, Comamonas sp., Azoarcus indigens, Chthoniobacter sp., Clostridium magnum, Lactobacillus harbinensis, Neobacillus drentensis, Clostridium methoxybenzovorans, Desulfovibrio sp., Colidextribacter sp., Comamonas aquatica, Lachnoclostridium sp., Clostridium saccharolyticum, Desulfosporosinus hippei, Sporomusa sp., Fonticella sp., Anaerovorax sp., Acinetobacter haemolyticus, Lactobacillus perolens, Desulfitobacterium sp., Mobilitalea sp., Desulfitobacterium dichloroeliminans, Achromobacter xylosoxidans, Clostridium sporosphaeroides, Acinetobacter sp., Rhodobacter sp., Lactobacillus bifermentans, Paenirhodobacter enshiensis, Hydrogenoanaerobacterium sp., Clostridium beijerinckii, Stenotrophomonas maltophilia, Elizabethkingia meningoseptica, Acinetobacter kookii, Stenotrophomonas acidaminiphila, Clostridium viride, Enterococcus casseliflavus, Sporomusa ovata, Desulfotomaculum defluvii, Pedobacter tournemirensis, Nitrosotenuis sp., Oscillibacter sp., Chryseobacterium taihuense, Ruminiclostridium sp., Ruminococcus sp., Anaerocolumna sp., Escherichia coli, Bacillus sp., Amnipila sp., Reyranella sp., Leuconostoc pseudomesenteroides, Comamonas terrigena, Citrobacter amalonaticus, Enterococcus italicus, Lactobacillus vaccinostercus, Butyricicoccus sp., Monoglobus sp., Kosakonia oryzae, Micropepsis sp., Clostridium tunisiense, Dechlorosoma sp., Ilyobacter delafieldii, Lacticaseibacillus sp., Udaeobacter sp., Delftia sp., Ruminiclostridium hungatei, Sporomusa silvacetica, Nitrosocosmicus oleophilus, Desulfotomaculum sp., Desulfosporomusa polytropa, Acidipropionibacterium acidipropionici, Caproiciproducens sp., Pandoraea pnomenusa, Actinomyces sp., Herbaspirillum huttiense, Phenylobacterium sp., Papillibacter sp., Saccharibacillus sp., Pseudomonas otitidis, Schaalia turicensis, Desulfosporosinus fructosivorans, Novosphingobium resinovorum, Lactobacillus delbrueckii, Pseudomonas putida, Anaerocolumna xylanovorans, Pseudomonas aeruginosa, Acinetobacter variabilis, Caulobacter sp., Clostridium subterminale, Acinetobacter ursingii, Oxobacter sp., Acetanaerobacterium sp., Clostridium intestinale, Lactobacillus pentosus, Sporomusa malonica, Rhodovastum sp., Clostridium homopropionicum, Paracoccus sp., Clostridium malenominatum, Anaerostignum sp., Aeromonas hydrophila, Desulfosporosinus meridiei, Acetobacter sp., Pseudarthrobacter oxydans, Azospirillum sp., Citrobacter sp., Pseudomonas stutzeri, Herbinix sp., Stenotrophomonas sp., Serratia marcescens, Acinetobacter radioresistens, Desulfosporosinus youngiae, Desulfofarcimen sp., Trabulsiella sp., Oxalophagus oxalicus, Clostridium tyrobutyricum, Enterococcus sp., Tabrizicola sp., Hydrogenophaga sp., Novosphingobium sp., Neorhizobium alkalisoli, Methylophilus sp., Desulfobulbus sp., Geminisphaera sp., Paludibacter sp., Solimonas flava, Flavobacterium sp., Pleomorphomonas sp., Sphingobium sp., Shewanella dokdonensis, Klebsiella pneumoniae, Rhizobium sp., Pseudacidovorax intermedius, Magnetospirillum sp., Siphonobacter aquaeclarae, Oleomonas sagaranensis, Riegeria sp., Quatrionicoccus sp., Paenirhodobacter sp., Ideonella dechloratans, Roseomonas sp., Xanthobacter polyaromaticivorans, Thiobacillus thioparus, Ancalomicrobium sp., Uliginosibacterium sp., Anaerosporobacter sp., Pseudomonas oleovorans, Devosia insulae, Agrobacterium tumefaciens, Erythrobacter mathurensis, Propionivibrio sp., Lacunisphaera sp., Chitinophaga pinensis, Xanthobacter autotrophicus, Mucilaginibacter litoreus, Dyadobacter fermentans, Brevundimonas aurantiaca, Bosea sp., Chitinophaga sancti, Xanthobacter flavus, Sphingobium yanoikuyae, Desulfovibrio vulgaris, Azoarcus olearius, Catellibacterium terrae, Rhodovarius lipocyclicus, Aquaspirillum polymorphum, Roseomonas rubra, Starkeya novella, Paracoccus yeei, Gracilibacter sp., Dechlorosoma suillum, Aeromonas sp., Acinetobacter johnsonii, Anaerotaenia torta, Parabacteroides chartae, Pedobacter rhizosphaerae, Anaerocolumna cellulosilytica, Rhizobium undicola, Shinella zoogloeoides, Bdellovibrio sp., Propionicimonas sp., Lachnotalea sp., Bosea thiooxidans, Variovorax ginsengisoli, Kaistia hirudinis, Zoogloea sp., Kaistia dalseonensis, Opitutus sp., Kaistia sp., Terrimicrobium sacchariphilum, Mycolicibacterium mucogenicum, Asticcacaulis sp., Rhizobium gallicum, Cellulosimicrobium sp., Prosthecobacter debontii, Mycobacterium gilvum, Sphingomonas wittichii, Cereibacter changlensis, Pseudomonas mendocina, Variovorax sp., Pseudaeromonas pectinilytica, Cupriavidus campinensis, Bacillus circulans, Fibrisoma sp., Moraxella osloensis, Ensifer adhaerens, Rhizobium rhizoryzae, Novosphingobium aromaticivorans, Shinella fusca, Roseomonas eburnea, Roseomonas cervicalis, Acinetobacter soli, Rhodoplanes roseus, Paenibacillus ihuae, Caulobacter mirabilis, Enterobacter sp., Flavobacterium akiainvivens, Pseudomonas alcaligenes, Paenibacillus graminis, Marinomonas sp., Xinfangfangia soli, Imtechium assamiensis, Raoultella planticola, Roseomonas aestuarii, Legionella lytica, Novosphingobium sediminicola, Yersinia pestis, Raoultella ornithinolytica, Enterobacter hormaechei, Mycoplana sp., Pseudomonas nitroreducens, Rhizobium petrolearium, Shinella kummerowiae, Propionispora vibrioides, Enterobacter cancerogenus, Ochrobactrum intermedium, Klebsiella sp., Acinetobacter tandoii, Cloacibacterium haliotis, Lactiplantibacillus sp., Yersinia bercovieri, Pelosinus sp., Acinetobacter lwoffii, Anaerospora sp., Lactobacillus coryniformis, Pseudomonas psychrotolerans, Agrobacterium albertimagni, Acinetobacter venetianus, Cyberlindnera jadinii, Penicillium herquei, Williopsis sp., Mortierella hyalina, Pichia kudriavzevii, Mucor circinelloides, Candida tropicalis, Preussia flanaganii, Mortierella sp., Penicillium sp., Penicillium melinii, Mortierella gamsii, Starmera stellimalicola, Purpureocillium lavendulum, Mucor irregularis, Monocillium indicum, Penicillium griseofulvum, Solicoccozyma aeria, Mortierella alpina, Monocillium mucidum, Mortierella ambigua, Tetracladium sp., Tausonia pullulans, Trichoderma piluliferum, Stropharia coronilla, Gymnostellatospora japonica, Aspergillus caninus, Meyerozyma caribbica, Penicillium bilaiae, Trichoderma harzianum, Pseudogymnoascus roseus, Penicillium oxalicum, Talaromyces marne ei, Phaeosphaeriopsis sp., Penicillium brevicompactum, Alternaria longissima, Candida parapsilosis, Pseudostrickeria sp., Allophoma tropica, Leptosphaeria maculans, Trichoderma pubescens, Cortinarius helvolus, Mortierella sarnyensis, Pyrenochaetopsis leptospora, Exophiala sp., Pseudothielavia terricola, Cladosporium herbarum, Trichoderma asperellum, Aspergillus fumigatus, Alternaria eichhorniae, Talaromyces piceae, Penicillium levitum, Talaromyces sp., Chrysosporium lobatum, Didymella exigua, Aspergillus chlamydosporus, Penicillium roseopurpureum, Subramaniula asteroides, Aspergillus pseudodeflectus, Penicillium citrinum, Chrysosporium pseudomerdarium, Mortierella rishikesha, Neobulgaria sp., Curvularia verruculosa, Clavaria sp., Plectosphaerella cucumerina, Mortierella minutissima, Penicillium parviverrucosum, Panaeolus fimicola, Psathyrella ichnusae, Arachnomyces gracilis, Solicoccozyma terrea, Mortierella antarctica, Coniothyrium cerealis, Neodactylaria sp., Penicillium pimiteouiense, Penicillium simplicissimum, Humicola olivacea, Arachnomyces kanei, Mortierella lignicola, Thelebolus globosus, Bipolaris microlaenae, Penicillium erubescens, Polyschema sclerotigenum, Phaeosphaeria sp., Sarocladium strictum, Penicillium restrictum, Fusarium proliferatum, Penicillium argentinense, Alternaria metachromatica, Coniothyrium sp., Talaromyces purpureogenus, Aspergillus niger, Lachnum sp., Gymnopus sp., Penicillium rubens, Alternaria photistica, Sporormiella megalospora, Didymella pinodes, Phallus rugulosus, Coniochaeta canina, Cutaneotrichosporon jirovecii, Aspergillus ochraceus, Penicillium catenatum, Microscypha sp., Sebacina sp., Talaromyces stollii, Oidiodendron cereale, Hannaella oryzae, Furcasterigmium furcatum, Fusarium solani, Clonostachys rosea, Aspergillus wentii, Kernia pachypleura, Olpidium brassicae, Penicillium steckii, Byssochlamys lagunculariae, Acrocalymma vagum, Cephalotrichum microsporum, Stemphylium vesicarium, Leohumicola verrucosa, Aspergillus subversicolor, Wallemia sebi, Trichosporon asahii, Rhizoctonia solani, Actinomucor elegans, Scedosporium dehoogii, Scedosporium boydii, Gibellulopsis serrae, Vishniacozyma victoriae, Cunninghamella echinulata, Chaetosphaeronema sp., Chrysosporium sp., Filobasidium magnum, Epicoccum thailandicum, Aspergillus sp., Oidiodendron echinulatum, Penicillium polonicum, Oedocephalum nayoroense, Fusarium oxysporum, Hirsutella minnesotensis, Aspergillus flavus, Penicillium atrovenetum, Chrysosporium synchronum, Hymenoscyphus menthae, Talaromyces radicus, Mortierella elongata, Microascus trigonosporus, Humicola fuscoatra, Vishniacozyma tephrensis, Aspergillus terreus, Neosetophoma samarorum, Aureobasidium pullulans, Wallemia canadensis, Circinella muscae, Botrytis caroliniana, Aspergillus penicillioides, Aspergillus rugulosus, Preussia sp., Malbranchea flocciformis, Malbranchea cinnamomea, Microdochium sp., Fusarium pseudensiforme, Chaetomium acropullum, Vishniacozyma globispora, or a combination thereof.

18. The method of claim 16, wherein the plant part comprises foliage, a stem, root, seedling, seed, or a combination thereof, and wherein the treated product comprises a treated soil, a treated plant, a treated plant tissue, or a treated plant part.

19. The method of claim 16, wherein the contacting lasts for between about 1 minute to about 24 hours.

20. The method of claim 16, wherein the period of time in between the repeating of the contacting step is between 24 hours and 12 weeks.
